(19) United States
(12) Patent Application Publication
(10) Pub. No.: US 2023/0235388 A1
(43) Pub. Date: Jul. 27, 2023

(54) COMPOSITIONS AND METHODS FOR SEQUENCING USING AT LEAST ALTERING ELECTRICAL CHARACTERISTICS OF BRIDGES BETWEEN ELECTRODES

(71) Applicant: Illumina Singapore PTE. LTD., Singapore (SG)

(72) Inventors: Hassan Bohra, Singapore (SG); Yin Nah Teo, Singapore (SG)

(73) Assignee: Illumina Singapore PTE. LTD., Singapore (SG)

(21) Appl. No.: 18/008,954

(22) PCT Filed: Jun. 24, 2021
(86) PCT No.: PCT/SG2021/050367

§ 371 Date: Dec. 7, 2022

Related U.S. Application Data
(60) Provisional application No. 63/046,663, filed on Jun. 30, 2020.

(51) Int. Cl. C12Q 1/6869; C12Q 1/6825

(52) U.S. Cl. CPC C12Q 1/6869; C12Q 1/6825

(57) ABSTRACT

Provided herein are compositions and methods for sequencing using at least altering electrical characteristics of polymer ridges. In some examples, the bridges may span the space between first and second electrodes and may include a single-stranded conjugated polymer chain. A plurality of nucleotides may be coupled to corresponding labels. A polymerase may be coupled to the bridge and may add nucleotides to a first polynucleotide using at least a sequence of a second polynucleotide. The labels corresponding to those nucleotides respectively may alter an electrical characteristic of the conjugated polymer chain. Detection circuitry may detect a sequence in which the polymerase adds the nucleotides to the first polynucleotide using at least changes in an electrical signal through the bridge, the changes being responsive to the respective alterations of hybridization using the labels corresponding to those nucleotides.

### CROSS-REFERENCE TO RELATED APPLICATIONS

This application claims the benefit of U.S. Provisional Patent Application No. 63/046,663, filed Jun. 30, 2020 and entitled “Compositions and Methods for Sequencing Using at Least Altering Electrical Characteristics of Bridges Between Electrodes,” the entire contents of which are incorporated by reference herein.

### BACKGROUND

A significant amount of academic and corporate time and energy has been invested into sequencing polynucleotides, such as DNA. Some sequencing systems use “sequencing by synthesis” (SBS) technology and fluorescence-based detection. However, fluorescence-based detection may require optical components such as excitation light sources, imaging devices, and the like, which may be complex, time-consuming to operate, and costly.

### SUMMARY

Examples provided herein are related to sequencing using at least altering electrical characteristics of bridges between electrodes. Compositions and methods for performing such sequencing are disclosed.

In some examples, a composition provided herein includes first and second electrodes separated from one another by a space, and a bridge spanning the space between the first and second electrodes. The bridge may include a single-stranded conjugated polymer chain. The composition further may include first and second polynucleotides, and a plurality of nucleotides, each nucleotide coupled to a corresponding label. The composition further may include a polymerase to add nucleotides of the plurality of nucleotides to the first polynucleotide using at least a sequence of the second polynucleotide. The labels corresponding to those nucleotides respectively may alter an electrical characteristic of the single-stranded conjugated polymer chain. The composition further may include detection circuitry to detect a sequence in which the polymerase adds the nucleotides to the first polynucleotide using at least changes in an electrical signal through the bridge, the changes being responsive to alteration of the electrical characteristic using the labels corresponding to those nucleotides.

In some examples, the single-stranded conjugated polymer chain includes a first delocalized set of orbitals, and each of the labels includes a respective delocalized set of orbitals that shares electrons with the first delocalized set of orbitals when the label is associated with the single-stranded polymer chain. In some examples, each of the labels and the single-stranded conjugated polymer chain forms a donor:acceptor complex. In some examples, the labels include respective acceptors, the single-stranded conjugated polymer chain includes a donor, and electrons transfer from the single-stranded polymer chain to each of the labels via π-π interactions. In some examples, the labels include different molecular orbitals and detectably change electrical conductivity of the single-stranded conjugated polymer chain differently.

In some examples, the single-stranded polymer chain includes a homopolymer. In some examples, the homopolymer includes repeating monomer units M1, each of the monomer units M1 being coupled to a solubilizing group R.

In other examples, the single-stranded polymer chain includes a copolymer including first monomer units M1 and second monomer units M2, each of the first monomer units M1 being coupled to a solubilizing group R. In some examples, the copolymer includes repeating M1-M2 units.

In still other examples, the single-stranded polymer chain includes a copolymer including first monomer units M1 and second monomer units M3, wherein (i) each of the first monomer units M1, or (ii) each of the second monomer units M3, or (iii) each of the first monomer units M1 and each of the second monomer units M3, is coupled to a solubilizing group R. In some examples, the copolymer includes repeating M1-M3 units.

In other examples, the single-stranded polymer chain includes a copolymer including first monomer units M3 and second monomer units M2, each of the first monomer units M3 being coupled to a solubilizing group R. In some examples, the copolymer includes repeating M3-M2 units.

In some examples, the single-stranded polymer chain includes a copolymer including first monomer units M1, second monomer units M2, and third monomer units M3, wherein (i) each of the first monomer units M1, or (ii) each of the second monomer units M3, or (iii) each of the first monomer units M1 and each of the second monomer units M3, is coupled to a solubilizing group R. In some examples, the copolymer includes repeating M1-M2 units. In some examples, the copolymer includes repeating M3-M2 units.

In some examples, M1 is an electron-donating or electron-neutral monomer unit that includes the solubilizing group R, and wherein the electron-donating or electron-neutral monomer unit that includes the solubilizing group R is selected from the group consisting of:

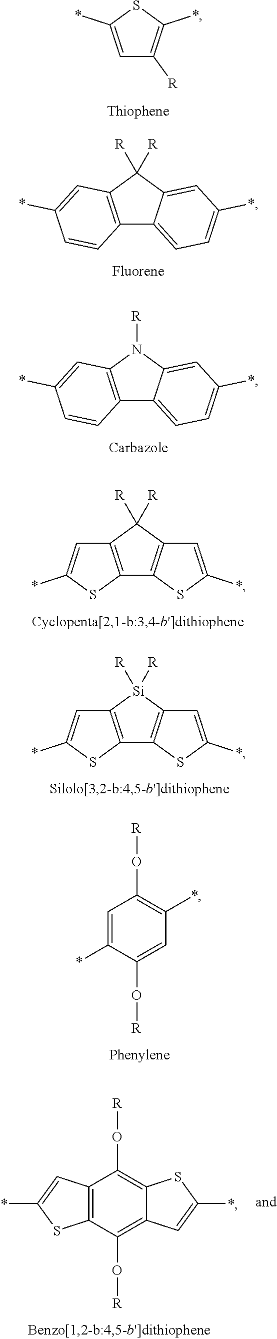

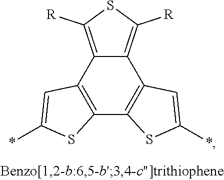

where * indicates coupling to an end group or to another monomer unit.

In some examples, R is a cationic solubilizing group selected from the group consisting of: (N,N,N-Trimethyl ammonium) alkyl, (N,N,N-Tributyl ammonium)alkyl, 1-methyl-3-(alkyl)-Imidazolium, and 1-(alkyl)Pyridinium.

In some examples, R is an anionic solubilizing group selected from the group consisting of: alkyl sulfonate, alkyl carboxylate, and alkyl phosphonate.

In some examples, M2 is an electron-donating monomer unit that excludes the solubilizing group R, and wherein the electron-donating monomer unit that excludes the solubilizing group R is selected from the group consisting of:

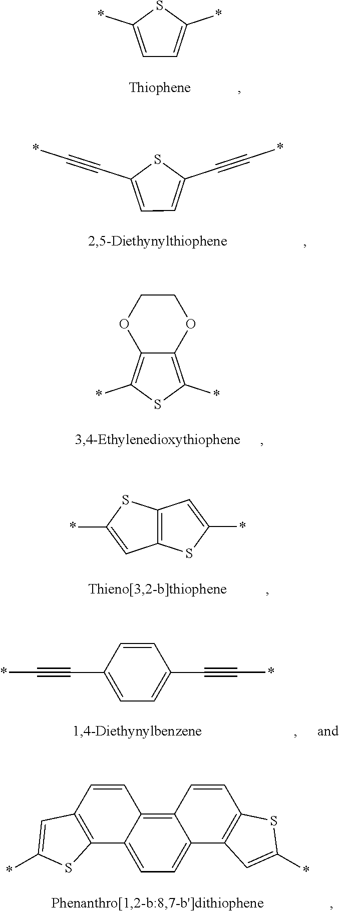

where * indicates coupling to an end group or to another monomer unit.

In some examples, M3 is an electron-withdrawing monomer unit that excludes the solubilizing group R, and wherein the electron-withdrawing monomer unit that excludes the solubilizing group R is selected from the group consisting of:

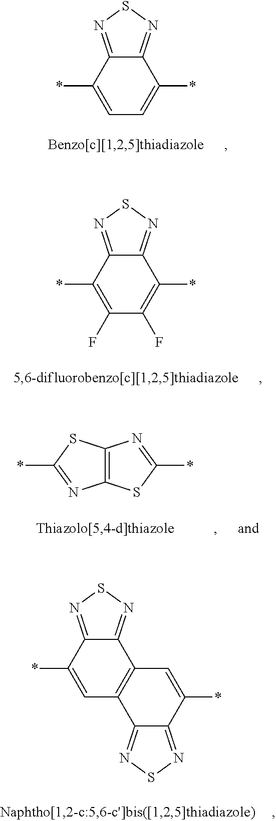

where * indicates coupling to an end group or to another monomer unit.

In some examples, M3 is an electron-withdrawing monomer unit that includes the solubilizing group R, and wherein the electron-withdrawing monomer unit that includes the solubilizing group R is selected from the group consisting of:

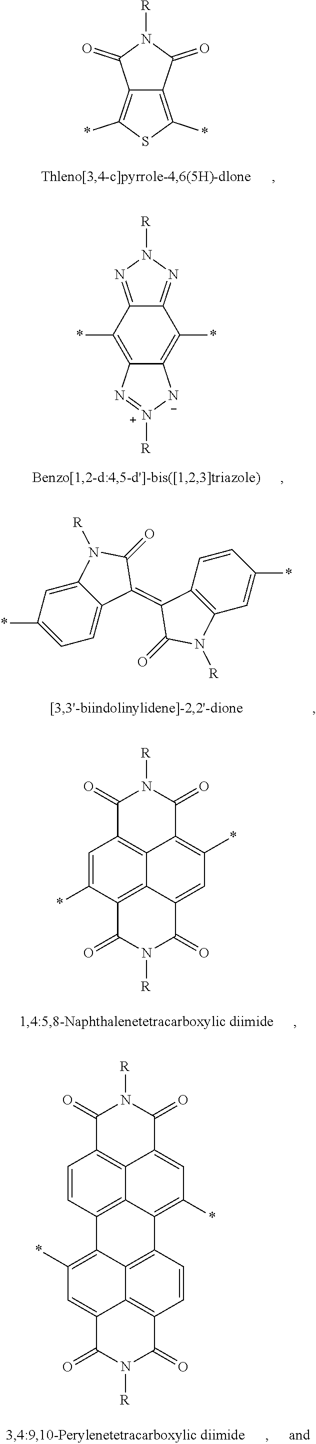

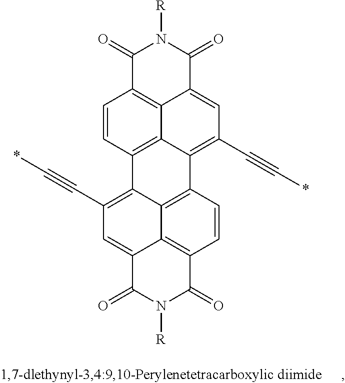

where * indicates coupling to an end group or to another monomer unit.

In some examples, the composition further includes a first end group coupling the single-stranded conjugated polymer chain to the first electrode, and a second end group coupling the single-stranded conjugated polymer to the second electrode. In some examples, each of the first and second end groups is selected from the group consisting of: Benzenethiol, Phenylacetylene, Benzenediazonium, Benzylamine, Methyl benzyl sulfide, and Methyl hexyl sulfide.

In some examples, the labels are acceptors selected from the group consisting of:

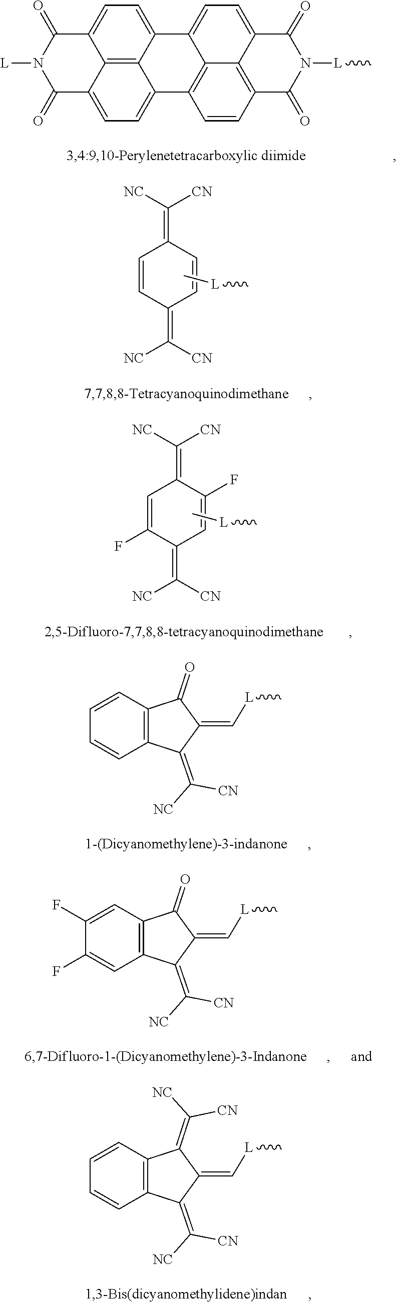

in which L indicates a linker and the wavy line represents coupling of linker L to the nucleotide.

A method is provided in some examples herein. The method includes adding, using a polymerase, nucleotides to a first polynucleotide using at least a sequence of a second polynucleotide. The method includes altering, using labels respectively coupled to the nucleotides, an electrical characteristic of a single-stranded conjugated polymer chain of a bridge spanning a space between first and second electrodes. The method includes detecting a sequence in which the polymerase adds the nucleotides to the first polynucleotide using at least changes in electrical signal through the bridge that are responsive to respective alterations of the electrical characteristic using the labels corresponding to those nucleotides.

In some examples, the single-stranded conjugated polymer chain includes a first delocalized set of orbitals, and each of the labels includes a respective delocalized set of orbitals that shares electrons with the first delocalized set of orbitals when the label is associated with the single-stranded polymer chain. In some examples, each of the labels and the single-stranded conjugated polymer chain forms a donor:acceptor complex. In some examples, the labels include respective acceptors, the single-stranded conjugated polymer chain includes a donor, and electrons transfer from the single-stranded polymer chain to each of the labels via π-π interactions. In some examples, the labels include different molecular orbitals and detectably change electrical conductivity of the single-stranded conjugated polymer chain differently.

In some examples, the single-stranded polymer chain includes a homopolymer. In some examples, the homopolymer includes repeating monomer units M1, each of the monomer units M1 being coupled to a solubilizing group R.

In other examples, the single-stranded polymer chain includes a copolymer including first monomer units M1 and second monomer units M2, each of the first monomer units M1 being coupled to a solubilizing group R. In some examples, the copolymer includes repeating M1-M2 units.

In still other examples, the single-stranded polymer chain includes a copolymer including first monomer units M1 and second monomer units M3, wherein (i) each of the first monomer units M1, or (ii) each of the second monomer units M3, or (iii) each of the first monomer units M1 and each of the second monomer units M3, is coupled to a solubilizing group R. In some examples, the copolymer includes repeating M1-M3 units.

In other examples, the single-stranded polymer chain includes a copolymer including first monomer units M3 and second monomer units M2, each of the first monomer units M3 being coupled to a solubilizing group R. In some examples, the copolymer includes repeating M3-M2 units.

In some examples, the single-stranded polymer chain includes a copolymer including first monomer units M1, second monomer units M2, and third monomer units M3, wherein (i) each of the first monomer units M1, or (ii) each of the second monomer units M3, or (iii) each of the first monomer units M1 and each of the second monomer units M3, is coupled to a solubilizing group R. In some examples, the copolymer includes repeating M1-M2 units. In some examples, the copolymer includes repeating M3-M2 units.

In some examples, M1 is an electron-donating or electron-neutral monomer unit that includes the solubilizing group R, and wherein the electron-donating or electron-neutral monomer unit that includes the solubilizing group R is selected from the group consisting of:

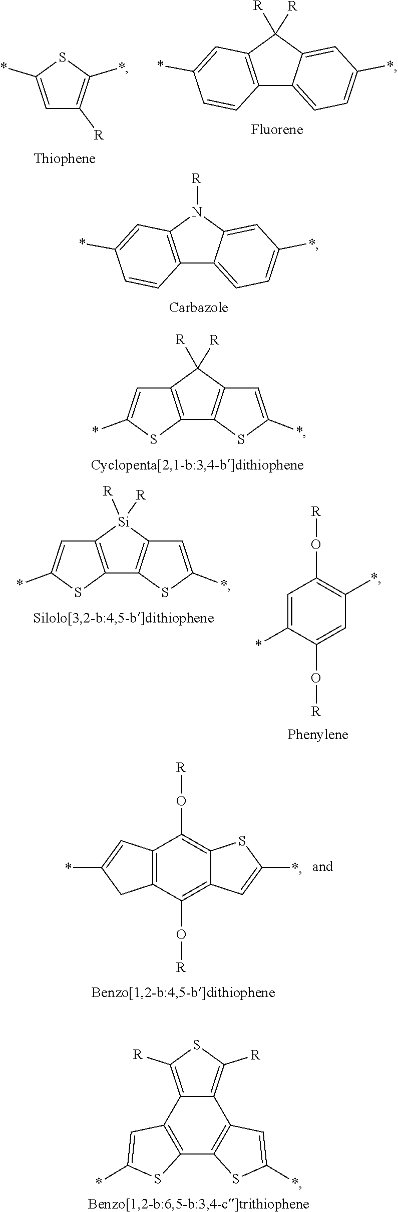

where * indicates coupling to an end group or to another monomer unit.

In some examples, R is a cationic solubilizing group selected from the group consisting of: (N,N,N-Trimethyl ammonium) alkyl, (N,N,N-Tributyl ammonium)alkyl, 1-methyl-3-(alkyl)-Imidazolium, and 1-(alkyl)Pyridinium.

In some examples, R is an anionic solubilizing group selected from the group consisting of: alkyl sulfonate, alkyl carboxylate, and alkyl phosphonate.

In some examples, M2 is an electron-donating monomer unit that excludes the solubilizing group R, and wherein the electron-donating monomer unit that excludes the solubilizing group R is selected from the group consisting of:

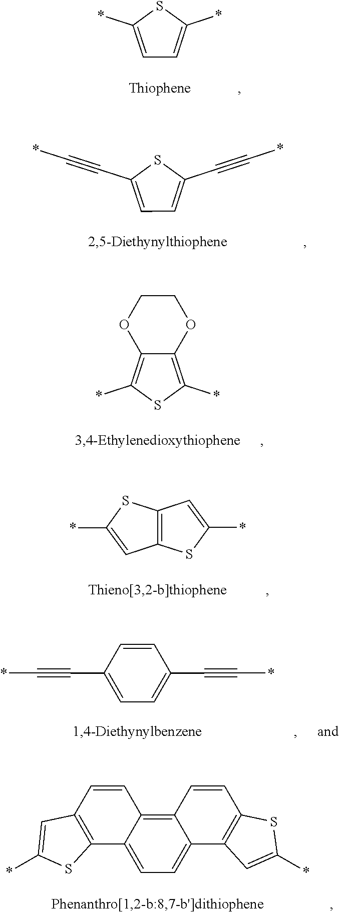

where * indicates coupling to an end group or to another monomer unit.

In some examples, M3 is an electron-withdrawing monomer unit that excludes the solubilizing group R, and wherein the electron-withdrawing monomer unit that excludes the solubilizing group R is selected from the group consisting of:

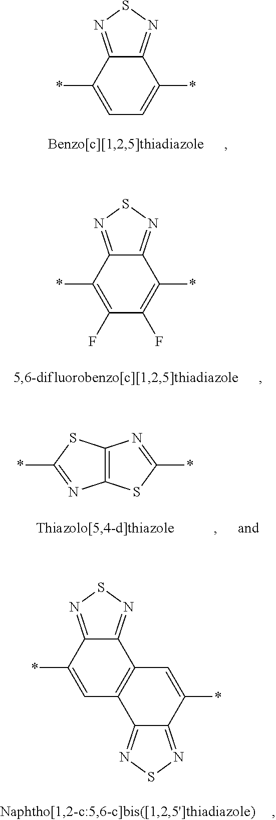

where * indicates coupling to an end group or to another monomer unit.

In some examples, M3 is an electron-withdrawing monomer unit that includes the solubilizing group R, and wherein the electron-withdrawing monomer unit that includes the solubilizing group R is selected from the group consisting of:

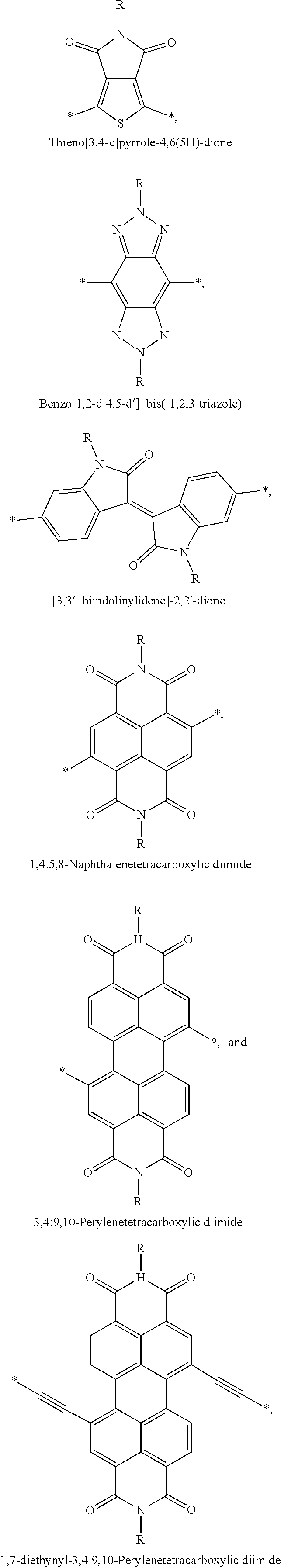

where * indicates coupling to an end group or to another monomer unit.

In some examples, the composition further includes a first end group coupling the single-stranded conjugated polymer chain to the first electrode, and a second end group coupling the single-stranded conjugated polymer to the second electrode. In some examples, each of the first and second end groups is selected from the group consisting of: Benzenethiol, Phenylacetylene, Benzenediazonium, Benzylamine, Methyl benzyl sulfide, and Methyl hexyl sulfide.

In some examples, the labels are acceptors selected from the group consisting of:

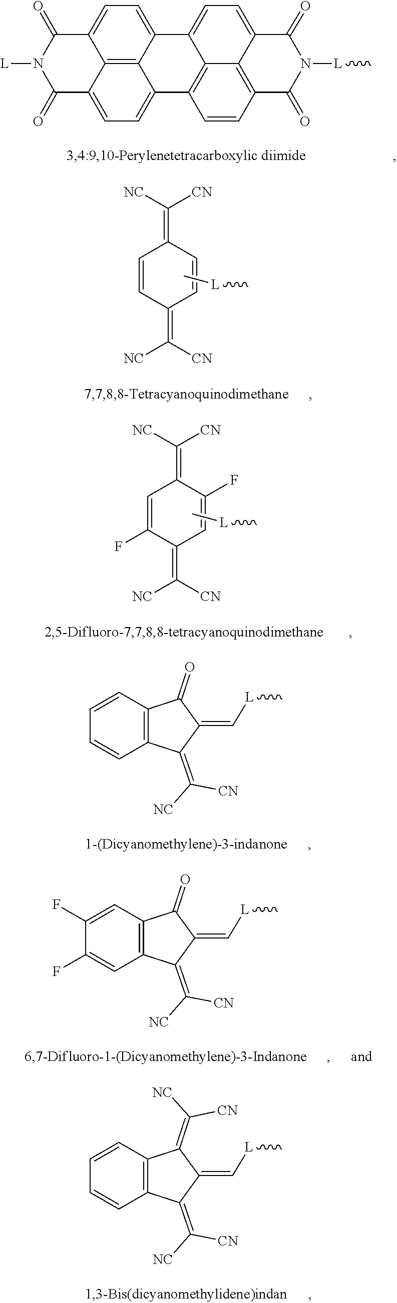

in which L indicates a linker and the wavy line represents coupling of linker L to the nucleotide.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1B schematically illustrate an example composition for sequencing that includes a single-stranded conjugated polymer bridge and labeled nucleotides that alter an electrical characteristic of the bridge.

FIGS. 2A-2E schematically illustrate example conjugated polymers that may be used for single-stranded polymer bridges.

FIG. 3A schematically illustrates example end groups that may be used for covalently coupling a single-stranded polymer bridge to an electrode.

FIG. 3B schematically illustrates example end groups that may be used for non-covalently coupling a single-stranded polymer bridge to an electrode.

FIG. 4 schematically illustrates an example acceptor-labeled nucleotide for use with a single-stranded conjugated polymer bridge.

FIG. 5 schematically illustrates example charge transfer schemes for altering an electrical characteristic of a single-stranded conjugated polymer bridge using an acceptor-labeled nucleotide.

FIG. 6 illustrates an example flow of operations in a method for sequencing using a single-stranded conjugated polymer bridge and labeled nucleotides that alter an electrical characteristic of the bridge.

### DETAILED DESCRIPTION

Examples provided herein are related to sequencing using at least altering electrical characteristics of polymer chains. Compositions and methods for performing such sequencing are disclosed.

More specifically, the present compositions and methods suitably may have the benefits of being used to sequence polynucleotides in a manner that is robust, reproducible, sensitive, accurate, works in real time, detects single molecules, and has high throughput. For example, the present compositions can include first and second electrodes and a bridge that spans the space between the electrodes. The bridge can include a single-stranded conjugated polymer chain that allows electrical current to flow from one electrode to another through the bridge. Labels, which may be coupled to respective nucleotides, may alter one or more electrical characteristics of the bridge, for example the electrical conductivity or electrical impedance of the bridge, and using at least such alteration the respective nucleotide may be identified.

First, some terms used herein will be briefly explained. Then, some example compositions and example methods for electronically sequencing polynucleotides will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially”, “approximately”, and “about” used throughout this Specification are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “electrode” is intended to mean a solid structure that conducts electricity. Electrodes may include any suitable electrically conductive material, such as gold, palladium, or platinum, or combinations thereof.

As used herein, the term “bridge” is intended to mean a structure that extends between, and attaches to, two other structures. A bridge may span a space between other structures, such as between two electrodes. Not all elements of a bridge need to be directly attached to both structures. However, some bridges directly attach to both structures. For example, in a bridge that includes a polymer chain spanning the space between two electrodes, one end of the polymer chain may attach directly to one of the electrodes, and the other end of the polymer chain may attach directly to the other electrode. A bridge may be attached to another structure, such as an electrode, via a chemical bond, e.g., via a covalent bond, hydrogen bond, ionic bond, dipole-dipole bond, London dispersion forces, or any suitable combination thereof.

As used herein, the term “single-stranded,” when referring to a bridge, is intended to mean that the bridge includes a single polymer strand.

As used herein, a “polymer” refers to a molecule including a chain of many subunits, that may be referred to as monomers, that are coupled to one another. The subunits may repeat, or may differ from one another. Some polymers are “conjugated,” which is intended to mean that the monomers constituting such polymers include a sufficient number of sp² hybridized atomic centers that sufficiently align with one another as to provide a delocalized set of orbitals via which electrons may flow along the length of the polymer. Nonlimiting examples of conjugated polymers, and their constituent monomers, are provided further below.

As used herein, a “donor” is intended to mean a molecule that is electron-rich, while an “acceptor” is intended to mean a molecule that is electron deficient. An “acceptor” may associate with (e.g., form a charge-transfer complex with) a “donor.” For example, an “acceptor” molecule that associates with a “donor” conjugated polymer in such a manner, forms a complex by withdrawing an electron from the polymer. This leads to an increase in free charge carrier concentration in the donor polymer and consequently an increase conductivity.

As used herein, “hybridize” is intended to mean noncovalently associating a first polymer to a second polymer along the lengths of those polymers. For instance, two DNA polynucleotide strands may associate through complementary base pairing. The strength of the association between the first and second polymers increases with the complementarity between the sequences of monomer units within those polymers. For example, the strength of the association between a first polynucleotide and a second polynucleotide increases with the complementarity between the sequences of nucleotides within those polynucleotides.

As used herein, the term “nucleotide” is intended to mean a molecule that includes a sugar and at least one phosphate group, and in some examples also includes a nucleobase. A nucleotide that lacks a nucleobase can be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide can be a single stranded sequence of nucleotides, such as RNA or single stranded DNA, a double stranded sequence of nucleotides, such as double stranded DNA, or can include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides can include non-naturally occurring DNA, such as enantiomeric DNA. The precise sequence of nucleotides in a polynucleotide can be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded polynucleotide template, and can sequentially add nucleotides to the growing primer to form a polynucleotide having a sequence that is complementary to that of the template.

As used herein, the term “primer” is defined as a polynucleotide to which nucleotides are added via a free 3′ OH group. A primer may have a 3′ block preventing polymerization until the block is removed. A primer can also have a modification at the 5′ terminus to allow a coupling reaction or to couple the primer to another moiety. The primer length can be any number of bases long and can include a variety of non-natural nucleotides.

As used herein, the term “label” is intended to mean a structure that attaches to a bridge in such a manner as to cause a change in an electrical characteristic of the bridge, such as electrical impedance or electrical conductivity, and based upon which change the nucleotide may be identified. For example, an acceptor-based label may non-covalently associate to a polymer chain within such a bridge, and the association may cause an electrical conductivity or electrical impedance change of the bridge. Or, for example, a polymer-based label may hybridize to a polymer chain within such a bridge, and the hybridization may cause an electrical conductivity or electrical impedance change of the bridge. However, it should be appreciated that a label may alter any suitable electrical characteristic of a polymer chain within a bridge. In examples provided herein, labels can be attached to nucleotides.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates can include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates can include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can comprise a single material or a plurality of different materials. Substrates can be composites or laminates. In some examples, the substrate comprises an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may comprise posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, Calif.).

Example Compositions and Methods for Sequencing Polynucleotides

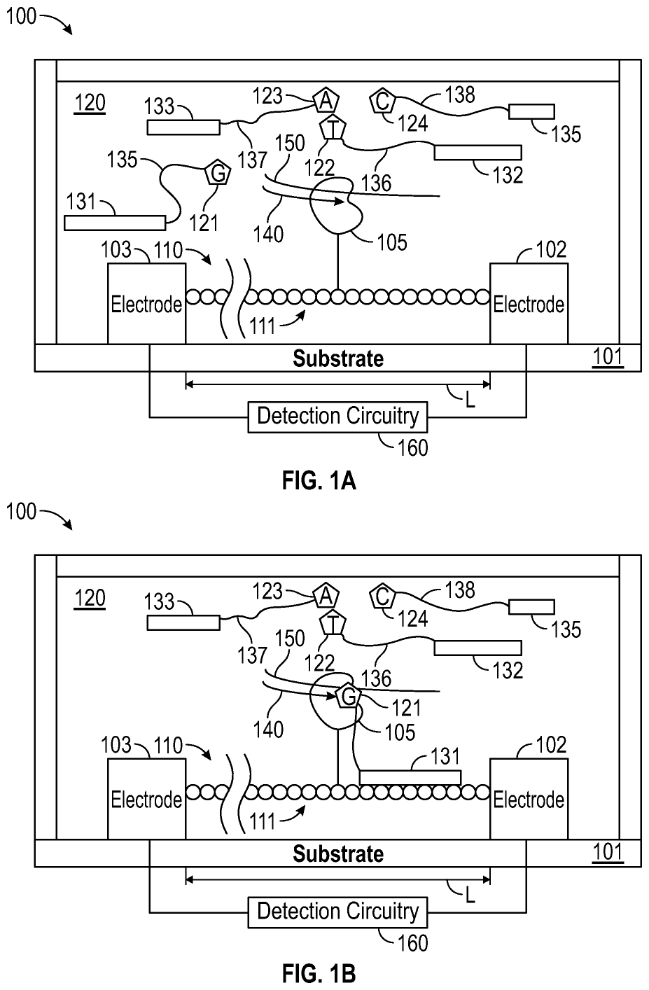

FIGS. 1A-1B schematically illustrate an example composition for sequencing that includes a single-stranded conjugated polymer bridge and labeled nucleotides that alter an electrical characteristic of the bridge. Referring now to FIG. 1A, composition 100 includes substrate 101, first electrode 102, second electrode 103, polymerase 104, bridge 110, nucleotides 121, 122, 123, and 124, labels 131, 132, 133, and 134 respectively coupled to those nucleotides, first polynucleotide 140, second polynucleotide 150, and detection circuitry 160. Polymerase 105 is in proximity of bridge 110, and in some examples may be coupled to bridge 110 via linker 106 in a manner such as known in the art. Such linker chemistries include maleimide chemistry to reactive thiols on cysteine residues, NHS ester chemistry to reactive amines on lysine residues, biotin-Streptavidin, and Spytag-SpyCatcher, for example. In the example illustrated in FIGS. 1A-1B, components of composition 100 may be enclosed within a flow cell (e.g., having walls 161, 162, 162) filled with fluid 120 in which nucleotides 121, 122, 123, and 124 (with associated labels), polynucleotides 140, 150, and suitable reagents may be carried.

Substrate 101 may support first electrode 102 and second electrode 103. First electrode 102 and second electrode 103 may be separated from one another by a space, e.g., a space of length L as indicated in FIG. 1A. The value of L may be, in some examples, from about 1 nm to about 1 μm, e.g., from about 1 nm to about 100 nm, e.g., from about 1 nm to about 10 nm, or e.g., from about 10 nm to about 25 nm, or e.g., from about 10 nm to about 30 nm, or, e.g., from about 25 nm to about 50 nm. First electrode 102 and second electrode 103 may have any suitable shape and arrangement, and are not limited to the approximately rectangular shape suggested in FIG. 1A. The sidewalls of first electrode 102 and second electrode 103 illustrated in FIG. 1A may be, but need not necessarily be, vertical or parallel to one another, and need not necessarily meet the top surfaces of such electrodes at a right angle. For example, first electrode 102 and second electrode 103 may be irregularly shaped, may be curved, or include any suitable number of obtuse or acute angles. In some examples, first electrode 102 and second electrode 103 may be arranged vertically relative to one another. The value L may refer to the spacing between the closest points of first electrode 102 and second electrode 103 to one another.

Bridge 110 may span the space between first electrode 102 and second electrode 103, and may include a single-stranded conjugated polymer chain 111 (the circles within the polymer chain being intended to suggest monomer units that are coupled to one another along the length of the polymer chain). Conjugated polymer chain 111 may have length that is approximately the same as length L of the space between first electrode 102 and second electrode 103 or otherwise permits polymer chain 111 to span the space between first electrode 102 and second electrode 103, e.g., such that polymer chain 111 may be attached directly to each of first electrode 102 and second electrode 103 (e.g., via respective bonds). It should be understood that in some configurations, polymer chain 111 need not necessarily be attached directly to one or both of first electrode 102 or second electrode 103. Instead, polymer chain 111 may be directly attached to one or more other structures that respectively are attached, directly or indirectly, to one or both of first electrode 102 and second electrode 103.

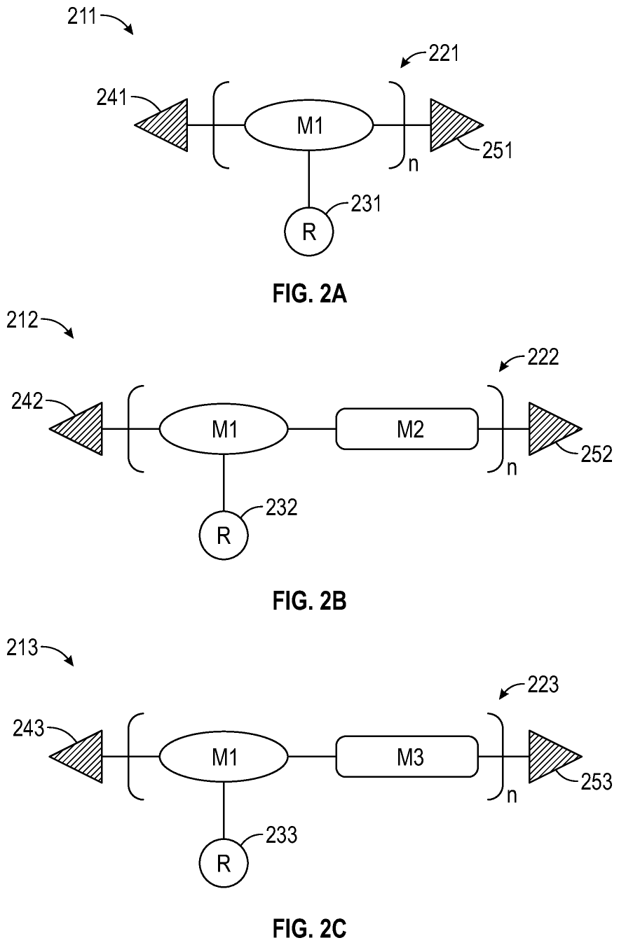

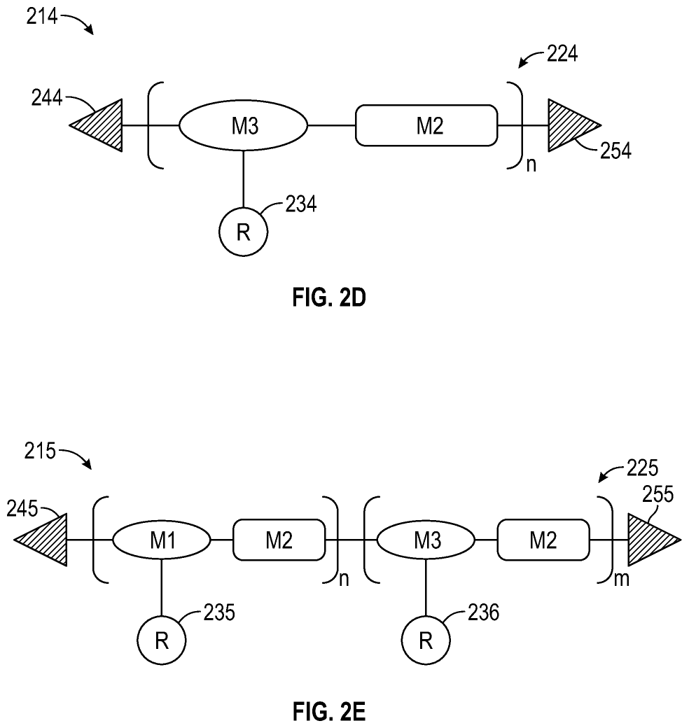

As explained in greater detail herein, labels 131, 132, 133, and 134 respectively may alter an electrical characteristic of single-stranded conjugated polymer chain 111 in such a manner as to modulate the electrical conductivity or impedance of bridge 110, based upon which modulation the identity of the corresponding nucleotides 121, 122, 123, and 124 may be determined. For example, as explained in greater detail with reference to FIG. 1B, labels 131, 132, 133, and 134 respectively may modulate the electrical conductivity or impedance of bridge 110, based upon which modulation the identity of the corresponding nucleotides 121, 122, 123, and 124 may be determined. For example, as described below with reference to FIGS. 2A-2E, FIGS. 3A-3B, FIG. 4, and FIG. 5, polymer chain 111 may be or include a conjugated polymer including a first delocalized set of orbitals. Labels 131, 132, 133, and 134 each may include a respective delocalized set of orbitals that, when associated with the conjugated polymer, share electrons with the first delocalized set of orbitals in such a manner as to alter electrical conductivity of the conjugated polymer. Illustratively, single-stranded conjugated polymer chain 111 and each of labels 131, 132, 133, 134 may form a corresponding charge transfer complex. For example, the conjugated polymer chain 111 may act as a donor and the labels 131, 132, 133, and 134 may act as respective acceptors. Each label 131, 132, 133, and 134 and the conjugated polymer chain 111 may form a donor:acceptor complex via π-π interactions, via which electrons may transfer from the donor to the acceptor so as to cause a change in electrical conductivity of the conjugated polymer chain. The extent of electron transfer from a donor to an acceptor depends on the extent of overlap between the molecular orbitals of the donor with the molecular orbitals of the acceptor. As such, labels which include different molecular orbitals may be expected to have different π-π interactions with the conjugated polymer chain, and as such to detectably change the electrical conductivity of the conjugated polymer chain differently, thus permitting detection circuitry 160 to uniquely identify the nucleotides to which such labels are coupled.

Composition 100 illustrated in FIG. 1A may include any suitable number of nucleotides coupled to corresponding labels, e.g., one or more nucleotides, two or more nucleotides, three or more nucleotides, or four nucleotides. For example, nucleotide 121 (illustratively, G) may be coupled to corresponding label 131, in some examples via linker 135. Nucleotide 122 (illustratively, T) may be coupled to corresponding label 132, in some examples via linker 136. Nucleotide 123 (illustratively, A) may be coupled to corresponding label 133, in some examples via linker 137. Nucleotide 124 (illustratively, C) may be coupled to corresponding label 134, in some examples via linker 138. The couplings between nucleotides and labels, in some examples via linkers which may include the same or different polymer as the labels, may be provided using any suitable methods known in the art, such as n-hydroxysuccinimide (NHS) ester chemistry or click chemistry.

As described below with reference to FIG. 4, labels 131, 132, 133, and 134 in some examples may include different acceptors that respectively may associate with (e.g., form charge-transfer complexes with) a conjugated polymer 111 in bridge 110 in such a manner as to reduce the flow of electrons through that conjugated polymer. In a manner such as described in greater detail with reference to FIG. 1B, the particular characteristics of labels 131, 132, 133, and 134 may be respectively selected so as to facilitate generation of distinguishable electrical signals, such as currents or voltages, through bridge 110 when those labels respectively alter an electrical characteristic of polymer chain 111. The labels may, but need not necessarily, alter the same electrical characteristic as one another. The labels may, but need not necessarily, alter the electrical characteristic of the same polymer chain as one another. For example, labels may alter different electrical characteristics of the polymer chain as one another, or may alter the same electrical characteristics of the same polymer chain as one another.

Composition 100 illustrated in FIG. 1A includes first polynucleotide 140 and second polynucleotide 150, and polymerase 105 that may add nucleotides of the plurality of nucleotides 121, 122, 123, and 124 to first polynucleotide 140 using at least a sequence of second polynucleotide 150. Detection circuitry 160 may detect a sequence in which polymerase 105 respectively adds the nucleotides 121, 122, 123, and 124 (not necessarily in that order) to first polynucleotide 140 using at least changes in a current through or impedance of bridge 110, the changes being responsive to the alterations in the electrical characteristic using the labels 131, 132, 133, and 134 corresponding to those nucleotides. For example, detection circuitry 160 may apply a voltage across first electrode 102 and second electrode 103, and may detect any current that flows through bridge 110 responsive to such voltage. Or, for example, detection circuitry 160 may flow a constant current through bridge 110, and detect a voltage difference between first electrode 102 and second electrode 103.

At the particular time illustrated in FIG. 1A, none of labels 131, 132, 133, and 134 is in contact with bridge 110, and so a baseline electrical (e.g., current) characteristic of the pristine polymer 111 may flow through bridge 110. Although nucleotides 121, 122, 123, 124 may diffuse freely through fluid 120 and respective labels 131, 132, 133, 134 may briefly contact bridge 110 as a result of such diffusion, the labels may relatively rapidly dissociate and so any resulting changes to the electrical conductivity or impedance of bridge 110 are expected to be so short as either to be undetectable, or to be clearly identifiable as not corresponding to addition of a nucleotide to first polynucleotide 140. For example, labels that associate as a result of diffusion or due to a polymerase-directed nucleotide incorporation may have identical hybridized lifetimes (statistically speaking). The lifetime is determined by the off rate of the interaction. The off rate is a constant that is governed by the nature of the interaction, temperature, salinity, buffer, and other factors. What distinguishes a true signal from a diffusive one is the percentage of time that the label is bound, and that is determined by the on rate. The on rate increases with the concentration of the label (in contrast to the off rate). For example, concentration corresponds to the probability of finding a molecule in a given volume. The concentration of the label can be orders of magnitude higher for bound nucleotides compared with diffusive ones, because the nucleotide is held in the active site. Thus, the on-rate is much higher. While the labels may dissociate equally fast in the diffusive and specific states, the specific state results in the labels reassociating relatively rapidly. After the nucleotide is incorporated, the linker between the label and the nucleotide is severed. Because the label may be coupled to the bridge via relatively weak electrostatic interactions, the next time the label dissociates, it has the same probability of floating away as the diffusive label.

In comparison, FIG. 1B illustrates a time at which polymerase 105 is adding nucleotide 121 (illustratively, G) to first polynucleotide 140 using at least the sequence of second polynucleotide 150 (e.g., so as to be complementary to a C in that sequence). Because polymerase 105 is acting upon nucleotide 121 to which label 131 is attached (in some examples via linker 137), such action maintains label 131 at a location that is sufficiently close to bridge 110 for a sufficient amount of time to alter an electrical characteristic of polymer chain 111 within alteration region 113, so as to cause a sufficiently long change in an electrical characteristic, such as electrical conductivity or impedance, of bridge 110 as to be detectable using detection circuitry 160, allowing identification of nucleotide 121 as being added to first polynucleotide 140. Additionally, label 131 may have a property that, when altering an electrical characteristic of polymer chain 111, an electrical characteristic, such as electrical conductivity or impedance, of bridge 110 via which detection circuitry 160 may uniquely identify the added nucleotide as 121 (illustratively G) as compared to any of the other nucleotides.

Similarly, label 132 may have a property that, when altering an electrical characteristic of polymer chain 111, alters an electrical characteristic, such as electrical conductivity or impedance, of bridge 110 via which detection circuitry 160 may uniquely identify the added nucleotide as 122 (illustratively T) as compared to any of the other nucleotides. Similarly, label 133 may have a property that, when altering an electrical characteristic of polymer chain 111, alters an electrical characteristic, such as electrical conductivity or impedance, of bridge 110 via which detection circuitry 160 may uniquely identify the added nucleotide as 123 (illustratively C) as compared to any of the other nucleotides. Similarly, label 134 may have a property that, when altering an electrical characteristic of polymer chain 111, alters an electrical characteristic, such as electrical conductivity or impedance, of bridge 110 via which detection circuitry 160 may uniquely identify the added nucleotide as 124 (illustratively C) as compared to any of the other nucleotides. It should be appreciated that labels 131, 132, 133, and 134 may have any suitable respective properties based upon which the electrical signal between first electrode 102 and second electrode 103 may vary in such a manner that detection circuitry 160 may identify nucleotides 121, 122, 123, 124 respectively coupled to those labels.

In some examples, the present conjugated polymer chains may provide for extended conjugation of π-electrodes over the polymer backbone. The conjugated polymer chains may include alternating electron-rich and electron-deficient aromatic molecules, leading to overlap of π-orbitals via which electrons may transfer along the polymer chain. The present polymer chains may include at least one building block, such as a monomer unit, that includes a pendant group that can be used to conjugate an electrolyte such as sulfonate, carboxylate, or quaternary amine to the polymer chain. Such groups, which may be referred to as solubilizing groups, may enhance the solubility of the polymer chain in an aqueous medium and may be introduced. Solubilizing groups may be coupled to the polymer chain, for example, using orthogonal click reactions via PEG linkers, or via direct conversion of a pendant group.

For example, FIGS. 2A-2E schematically illustrate example conjugated polymers that may be used for single-stranded polymer bridges. It will be appreciated that any of the conjugated polymers described with reference to FIGS. 2A-2E are non-limiting examples that may be used for bridge 110 described with reference to FIGS. 1A-1B, and that other conjugated polymers suitably may be used for bridge 110. In some examples, the backbones of conjugated polymers such as described with reference to FIGS. 2A-2E may be prepared using Suzuki-Miyaura polymerization.

Referring now to FIG. 2A, conjugated polymer 211 includes backbone 221, a plurality of solubilizing groups 231 coupled to backbone 221, and first and second end groups 241, 251 coupled to respective ends of backbone 221. In this example, backbone 221 includes a plurality of monomer units M1 that are polymerized with one another. As such, conjugated polymer 211 may be referred to as a “homopolymer” of M1. A respective solubilizing group R 231 may be coupled to each of the monomer units M1, e.g., so that the conjugated polymer 211 includes about n solubilizing groups R corresponding to about n monomer units M1. First end group 241 may include a first functional group that may be coupled to backbone 221 (e.g., via a first terminal monomer unit M1 of backbone 221) and may be coupled directly or indirectly to first electrode 102. Second end group 251 may include a second functional group that may be coupled to backbone 221 (e.g., via a second terminal monomer unit M1 of backbone 221) and may be coupled directly or indirectly to second electrode 103. In some examples, first and second end groups 241, 251 may be the same as one another. Conjugated polymer 211 may include a sufficient number of polymerized monomer units M1 as to substantially span the entire space between the first and second electrodes 102, 103. That is, the length of conjugated polymer 211, including the backbone and first and second end groups, may be at least of length L. Illustratively, the value of n for conjugated polymer 211 may be in the range of about 10 to about 50, or about 50 to about 100, or about 100 to about 150, or about 150 to about 200, or about 200 to about 300. Nonlimiting examples of monomer units M1, solubilizing groups R, and end groups are provided further below.

Conjugated polymer 212 illustrated in FIG. 2B includes backbone 222, a plurality of solubilizing groups 232 coupled to backbone 222, and first and second end groups 242, 252 coupled to respective ends of backbone 222. In this example, backbone 222 includes a plurality of monomer units M1 that are polymerized with a plurality of monomer units M2 that are different than M1. As such, conjugated polymer 212 may be referred to as an “alternating copolymer” of M1 and M2. Illustratively, conjugated polymer 212 may include a plurality of repeating M1-M2 subunits, e.g., about n repeating M1-M2 subunits. A respective solubilizing group R 232 may be coupled to each of the monomer units M1, e.g., so that the conjugated polymer 212 includes about n solubilizing groups corresponding to about n M1-M2 subunits. First end group 242 may include a first functional group that may be coupled to backbone 222 (e.g., via a terminal monomer unit M1 of backbone 222) and may be coupled directly or indirectly to first electrode 102. Second end group 252 may include a second functional group that may be coupled to backbone 222 (e.g., via a terminal monomer unit M2 of backbone 222) and may be coupled directly or indirectly to second electrode 103. In some examples, first and second end groups 242, 252 may be the same as one another. Conjugated polymer 212 may include a sufficient number of polymerized monomer units M1, M2 as to substantially span the entire space between the first and second electrodes 102, 103. That is, the length of conjugated polymer 212, including the backbone and first and second end groups, may be at least of length L. Illustratively, the value of n for conjugated polymer 212 may be in the range of about 10 to about 50, or about 50 to about 100, or about 100 to about 150, or about 150 to about 200, or about 200 to about 300. Nonlimiting examples of monomer units M1 and M2, solubilizing groups R, and end groups are provided further below.

Conjugated polymer 213 illustrated in FIG. 2C includes backbone 223, a plurality of solubilizing groups 233 coupled to backbone 223, and first and second end groups 243, 253 coupled to respective ends of backbone 223. In this example, backbone 223 includes a plurality of monomer units M1 that are polymerized with a plurality of monomer units M3 that are different than M1. As such, conjugated polymer 213 may be referred to as an “alternating copolymer” of M1 and M3. Illustratively, conjugated polymer 213 may include a plurality of repeating M1-M3 subunits, e.g., about n repeating M1-M3 subunits. Each of the monomer units M1, or each of the monomer units M3, or each of the monomer units M1 and of the second monomer units M3 may be coupled to a solubilizing group R. For example, a respective solubilizing group R 233 may be coupled to each of the monomer units M1, e.g., so that the conjugated polymer 213 includes about n solubilizing groups corresponding to about n M1-M3 subunits. Alternatively, a respective solubilizing group R 233 may be coupled to each of the monomer units M3, e.g., so that the conjugated polymer 213 includes about n solubilizing groups corresponding to about n M1-M3 subunits. As a further alternative, a respective solubilizing group R 233 may be coupled to each of the monomer units M1 and to each of the monomer units M3, e.g., so that the conjugated polymer 213 includes about 2n solubilizing groups corresponding to about n M1-M3 subunits. First end group 243 may include a first functional group that may be coupled to backbone 223 (e.g., via a terminal monomer unit M1 of backbone 223) and may be coupled directly or indirectly to first electrode 102. Second end group 253 may include a second functional group that may be coupled to backbone 223 (e.g., via a terminal monomer unit M3 of backbone 223) and may be coupled directly or indirectly to second electrode 103. In some examples, first and second end groups 243, 253 may be the same as one another. Conjugated polymer 213 may include a sufficient number of polymerized monomer units M1, M3 as to substantially span the entire space between the first and second electrodes 102, 103. That is, the length of conjugated polymer 213, including the backbone and first and second end groups, may be at least of length L. Illustratively, the value of n for conjugated polymer 213 may be in the range of about 10 to about 50, or about 50 to about 100, or about 100 to about 150, or about 150 to about 200, or about 200 to about 300. Nonlimiting examples of monomer units M1 and M3, solubilizing groups R, and end groups are provided further below.

Conjugated polymer 214 illustrated in FIG. 2D includes backbone 224, a plurality of solubilizing groups 234 coupled to backbone 224, and first and second end groups 244, 254 coupled to respective ends of backbone 224. In this example, backbone 224 includes a plurality of monomer units M3 that are polymerized with a plurality of monomer units M2 that are different than M3. As such, conjugated polymer 214 may be referred to as an “alternating copolymer” of M3 and M2. Illustratively, conjugated polymer 214 may include a plurality of repeating M3-M2 subunits, e.g., about n repeating M3-M2 subunits. A respective solubilizing group R 234 may be coupled to each of the monomer units M3, e.g., so that the conjugated polymer 214 includes about n solubilizing groups corresponding to about n M3-M2 subunits. First end group 244 may include a first functional group that may be coupled to backbone 224 (e.g., via a terminal monomer unit M3 of backbone 224) and may be coupled directly or indirectly to first electrode 102. Second end group 254 may include a second functional group that may be coupled to backbone 224 (e.g., via a terminal monomer unit M2 of backbone 224) and may be coupled directly or indirectly to second electrode 103. In some examples, first and second end groups 244, 254 may be the same as one another. Conjugated polymer 214 may include a sufficient number of polymerized monomer units M3, M2 as to substantially span the entire space between the first and second electrodes 102, 103. That is, the length of conjugated polymer 214, including the backbone and first and second end groups, may be at least of length L. Illustratively, the value of n for conjugated polymer 214 may be in the range of about 10 to about 50, or about 50 to about 100, or about 100 to about 150, or about 150 to about 200, or about 200 to about 300. Nonlimiting examples of monomer units M3 and M2, solubilizing groups R, and end groups are provided further below.

Conjugated polymer 215 illustrated in FIG. 2E includes backbone 225, a plurality of solubilizing groups 235, 236 coupled to respective portions of backbone 225, and first and second end groups 245, 255 coupled to respective ends of backbone 225. In this example, backbone 225 includes a plurality of monomer units M1, M2, M3 that are different. As such, conjugated polymer 215 may be referred to as a “random copolymer” of M1, M2, and M3. Illustratively, conjugated polymer 215 may include a plurality of repeating M1-M2 subunits, e.g., about n repeating M1-M2 subunits, and a plurality of repeating M3-M2 subunits, e.g., about m repeating M3-M2 subunits. The plurality of M1-M2 subunits may be coupled to the plurality of M3-M2 subunits. For example, a terminal monomer unit M2 of the plurality of M1-M2 subunits may be coupled to a terminal monomer unit M3 of the plurality of M3-M2 subunits. In some examples, (i) each of the monomer units M1, or (ii) each of the monomer units M3, or (iii) each of the monomer units M1 and each of the monomer units M3, may be coupled to a solubilizing group R. For example, a respective solubilizing group R 235 may be coupled to each of the monomer units M1, e.g., so that the conjugated polymer 215 includes about n solubilizing groups corresponding to about n M1-M2 subunits. Additionally, or alternatively, respective solubilizing group R 236 may be coupled to each of the monomer units M3, e.g., so that the conjugated polymer 215 includes about m solubilizing groups corresponding to about m M3-M2 subunits. Solubilizing groups 235, 236 may be of the same type as one another, or may be different types. First end group 245 may include a first functional group that may be coupled to backbone 225 (e.g., via a terminal monomer unit M1 of backbone 225) and may be coupled directly or indirectly to first electrode 102. Second end group 255 may include a second functional group that may be coupled to backbone 225 (e.g., via a terminal monomer unit M2 of backbone 225) and may be coupled directly or indirectly to second electrode 103. In some examples, first and second end groups 245, 255 may be the same as one another. Conjugated polymer 215 may include a sufficient number of polymerized monomer units M1, M2, M3 as to substantially span the entire space between the first and second electrodes 102, 103. That is, the length of conjugated polymer 215, including the backbone and first and second end groups, may be at least of length L. Illustratively, the value of n for conjugated polymer 215 may be in the range of about 10 to about 50, or about 50 to about 100, or about 100 to about 150, or about 150 to about 200, or about 200 to about 300, and the value of m may be in the range of about 10 to about 50, or about 50 to about 100, or about 100 to about 150, or about 150 to about 200, or about 200 to about 300. Nonlimiting examples of monomer units M1, M2, and M3, solubilizing groups R, and end groups are provided further below.

In examples such as described with reference to FIGS. 2A-2C and 2E, monomer unit M1 may, purely by way of example, be an electron-donating or electron-neutral monomer unit that includes one or more solubilizing groups. Illustratively, the electron-donating or electron-neutral monomer unit that includes the one or more solubilizing groups may be selected from the group consisting of: Thiophene, Fluorene, Carbazole, Cyclopenta[2,1-b:3,4-b′]dithiophene, Silolo[3,2-b:4,5-b′]dithiophene, Phenylene, Benzo[1,2-b:4,5-b′]dithiophene (which may be referred to as Benzodithiophene), and Benzo[1,2-b:6,5-b′3,4-c′]trithiophene, example structures for which are shown below:

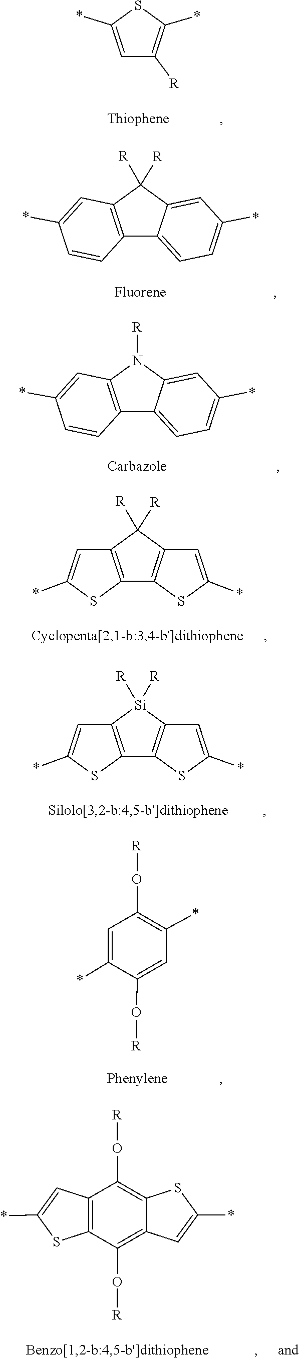

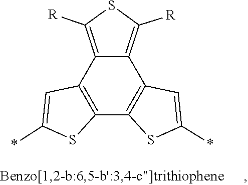

in which * indicates coupling of the monomer to another element (e.g., to an end group or another monomer unit) and R indicates the solubilizing group.

The solubilizing groups R of the polymer chain may be selected so as to provide suitable solubility of the polymer chain in an aqueous medium. In examples such as described with reference to FIGS. 2A-2E, solubilizing group R may, purely by way of example, be a charged group, such as a cationic group or an anionic group. Illustratively, the cationic solubilizing group may be selected from the group consisting of: (N,N,N-Trimethyl ammonium) alkyl, (N,N,N-Tributyl ammonium)alkyl, 1-methyl-3-(alkyl)-Imidazolium, and 1-(alkyl)Pyridinium. Illustratively, the anionic solubilizing group may be selected from the group consisting of: alkyl sulfonate, alkyl carboxylate, and alkyl phosphonate. Illustratively, the value of p for solubilizing group R may be in the range of about 2 to about 4, or about 4 to about 8, or about 8 to about 12. Example structures for such cationic solubilizing groups are shown below:

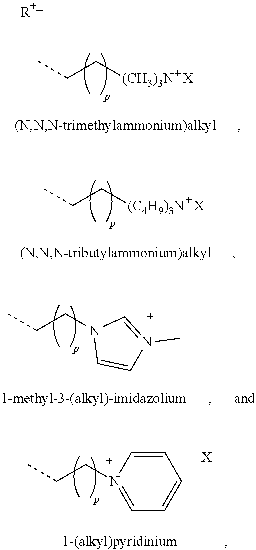

where X is a counter ion (X=Cl⁻, Br⁻, or I⁻).

Example structures for such anionic solubilizing groups are shown below:

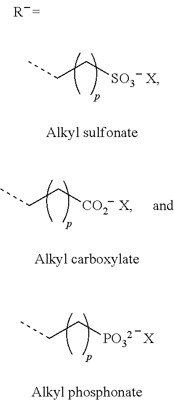

where X is a counter ion (X=H⁺, Na⁺, K⁺, or (n-C₂H₉)₄N⁺).

In examples such as described with reference to FIGS. 2B, 2D, and 2E, monomer unit M2 may be an electron-donating monomer unit that excludes a solubilizing group. Purely by way of example, the electron-donating monomer unit that excludes a solubilizing group may be selected from the group consisting of: Thiophene, 2,5-diethynylthiophene, 3,4-ethylenedioxythiophene, thieno[3,2-b]thiophene, 1,4-diethynylbenzene, and phenanthro[1,2-b:8,7-b]dithiophene, example structures for which are shown below:

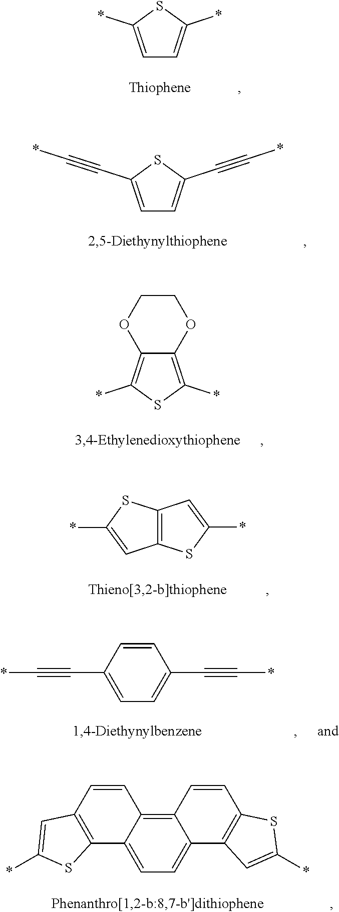

in which * indicates coupling of the monomer to another element (e.g., to an end group or another monomer unit) and R indicates the solubilizing group.

In examples such as described with reference to FIGS. 2C-2E, monomer unit M3 may be an electron-withdrawing monomer unit that includes one or more solubilizing groups, or that excludes a solubilizing group. Purely by way of example, the electron-withdrawing monomer unit that excludes a solubilizing group may be selected from the group consisting of: Benzo[c][1,2,5]thiadiazole, 5,6-difluorobenzo[c][1,2,5]thiadiazole, Thiazolo[5,4-d]thiazole, and Naphtho[1,2-c:5,6-c′]bis([1,2,5]thiadiazole). Alternatively, the electron-withdrawing monomer unit that includes one or more solubilizing groups may be selected from the group consisting of: Thieno[3,4-c]pyrrole-4,6(5H)-dione, [3,3′-biindolinylidene]-2,2′-dione, 1,4:5,8-Naphthalenetetracarboxylic diimide, 3,4:9,10-Perylenetetracarboxylic diimide, and 1,7-diethynyl-3,4:9,10-Perylenetetracarboxylic diimide. Example structures for such electron-withdrawing monomers that include one or more solubilizing groups are provided below:

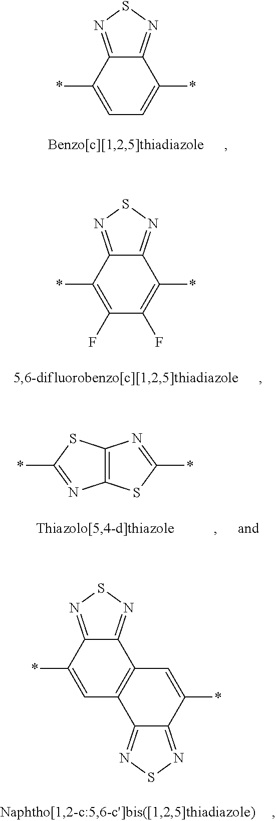 [SMILES: *c1ccc(*)c2nsnc12 ; *c1c(F)c(F)c(*)c2nsnc12 ; *c1nc2sc(*)nc2s1 ; *c1cc2c(cc(*)c3nsnc32)c2nsnc12]

where * indicates coupling to another monomer unit or to an end group. Example structures for such electron-withdrawing monomers that include one or more solubilizing groups are provided below:

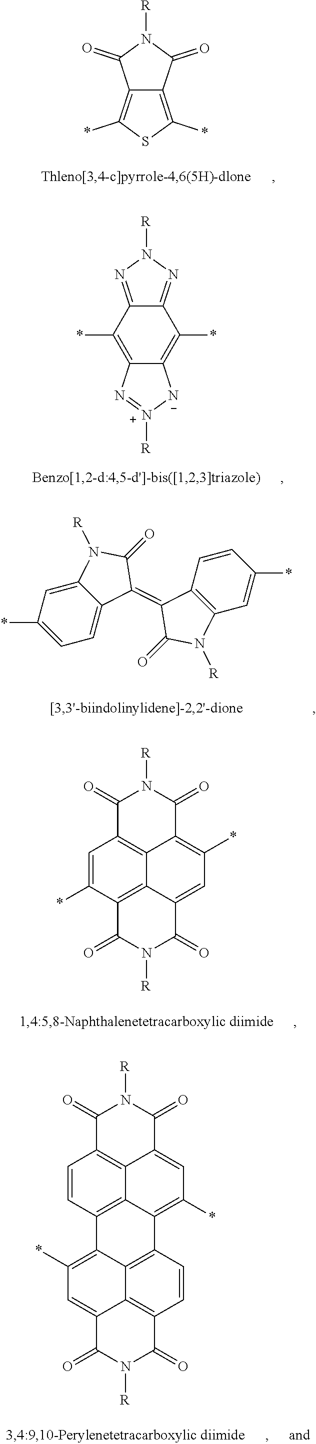

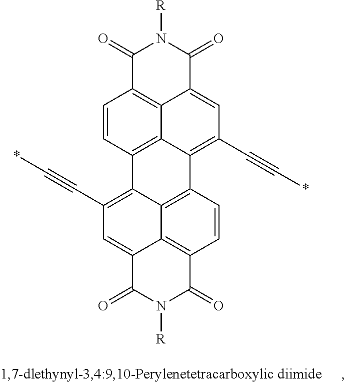

where * indicates coupling to another monomer unit or to an end group.

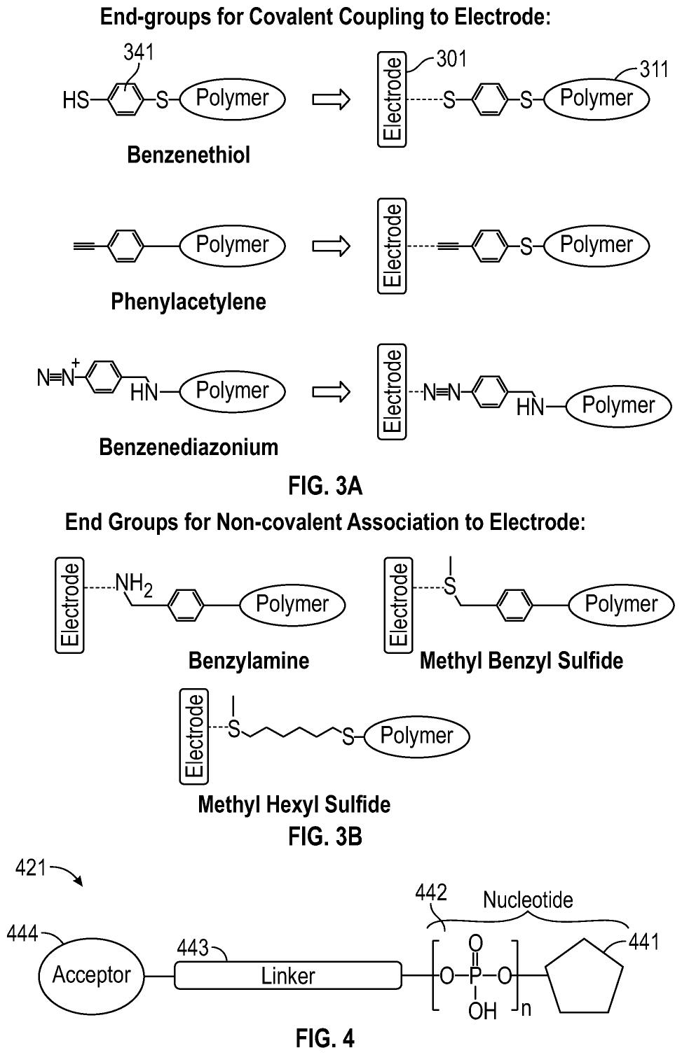

The end groups of the polymer chain suitably may be selected so as to provide attachment to respective electrodes. In some examples, the end groups covalently bond the polymer chains to the electrodes. In some examples, the end groups non-covalently bond the polymer chains to the electrodes, e.g., the polymer chains are weakly physisorbed to the electrodes via the end groups. In examples such as described with reference to FIGS. 2A-2E, each of the first and second end groups may, purely by way of example, be selected from the group consisting of: Benzenethiol, Phenylacetylene, Benzenediazonium, Benzylamine, Methyl benzyl sulfide, and Methyl hexyl sulfide. FIG. 3A schematically illustrates example end groups that may be used for covalently coupling a single-stranded polymer bridge to an electrode, and FIG. 3B schematically illustrates example end groups that may be used for non-covalently coupling a single-stranded polymer bridge to an electrode, in which example reactions between the end groups 341 and metal electrodes 301 are shown in dotted lines, and in which couplings between the end groups and the polymer 311 are shown in solid lines. Illustratively, in the non-limiting examples shown in FIG. 3A, for Benzenethiol, the thiol moiety may react with the metal electrode to form a covalent coupling; for Phenylacetylene, the acetylene moiety may react with the metal electrode to form a covalent coupling; and for Benzenediazonium, the diazonium moiety may react with the metal electrode to form a covalent coupling. In other non-limiting examples such as shown in FIG. 3B, for Benzylamine, the amine moiety may associate with the metal electrode to form a noncovalent coupling; for Methyl benzyl sulfide, the methyl sulfide moiety may associate with the metal electrode to form a noncovalent coupling; and for Methyl hexyl sulfide, the methyl sulfide moiety may associate with the metal electrode to form a noncovalent coupling.

It will be appreciated that any suitable labels 131, 132, 133, 134 may be selected so as to modulate the conduction of electron flow by a single-stranded conjugated polymer bridge such as described with reference to FIGS. 1A-1B, 2A-2E, and FIGS. 3A-3B. FIG. 4 schematically illustrates an example acceptor-labeled nucleotide 421 for use with a single-stranded conjugated polymer bridge. Acceptor-labeled nucleotide 421 may include nucleotide 441 coupled to any suitable number of phosphate groups 442, linker 443, and acceptor 444. Linker 443 couples acceptor 444 to a terminal one of the phosphate groups 442. Example nucleotides, phosphate groups, and linkers are described elsewhere herein. Purely by way of example, the acceptors may be selected from the group consisting of: 3,4:9,10-Perylenetetracarboxylic diimide, 7,7,8,8-Tetracyano quinodimethane, 2,5-Difluoro-7,7,8,8-tetracyano quinodimethane, 1-(Dicyanomethylene)-3-indanone, 6,7-Difluoro (Dicyanomethylene)-3-indanone, and 1,3-Bis(dicyanomethylidene)indan, example structures for which are shown below:

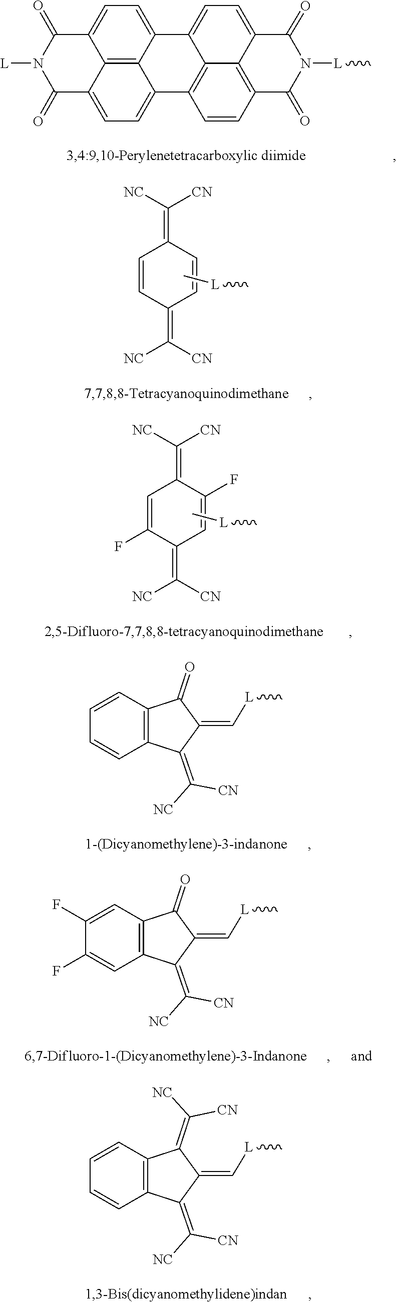

in which L indicates a linker and the wavy line represents coupling of linker L to the nucleotide.

The linkers L of the labeled nucleotides may be selected so as to provide suitable solubility of the labeled nucleotides in an aqueous medium and so as to enhance ionic interactions with the polymer chain. For example, to overcome the inhibitory effect of bulky acceptor labels on incorporation of nucleotides using a DNA polymerase, the acceptor may be distanced from the nucleotide using a relatively long linker, e.g., a linker including a relatively long PEG group. Additionally, or alternatively, the linker may be charged. Such a linker may include one or more charged groups such as or one or more pH-dependent polyelectrolytes, e.g., polylysines. Such a linker may also include a combination of PEG groups and pH-dependent polyelectrolytes. Alternatively, such a linker may include a relatively long polypeptide chain containing a combination of neutral and charged amino acids. Electrostatic interactions between the electrolytes coupled to the conjugated polymer chain and electrolytes on the linker of the labeled nucleotide may help to align the molecular orbitals of the conjugated polymer chain with those of the label, thereby facilitating charge-transfer complex formation. Purely by way of example, the linkers L that may be used to couple acceptors (such as those listed above) to nucleotides may be selected from the group consisting of: Polyethylene glycol, polyalanine, polyglycine, polylysine, and polyglutamic acid, example structures for which are shown below:

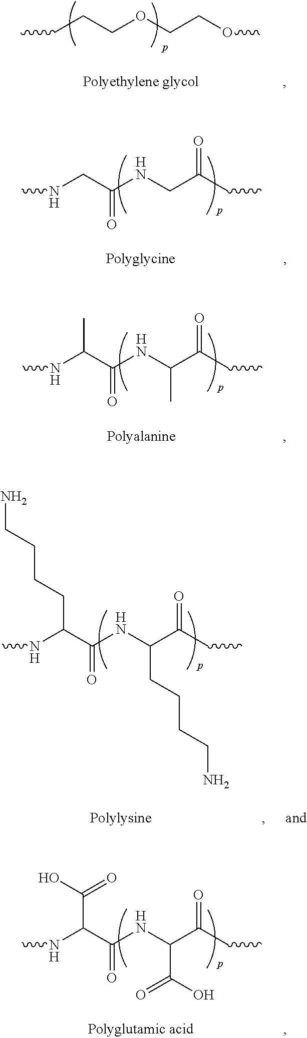

in which the wavy lines indicate respective couplings to the acceptor and to the nucleotide. Illustratively, the value of p for linker L may be in the range of about 2 to about 4, or about 4 to about 8, or about 8 to about 12, or about 12 to about 14, or about 14 to about 16.

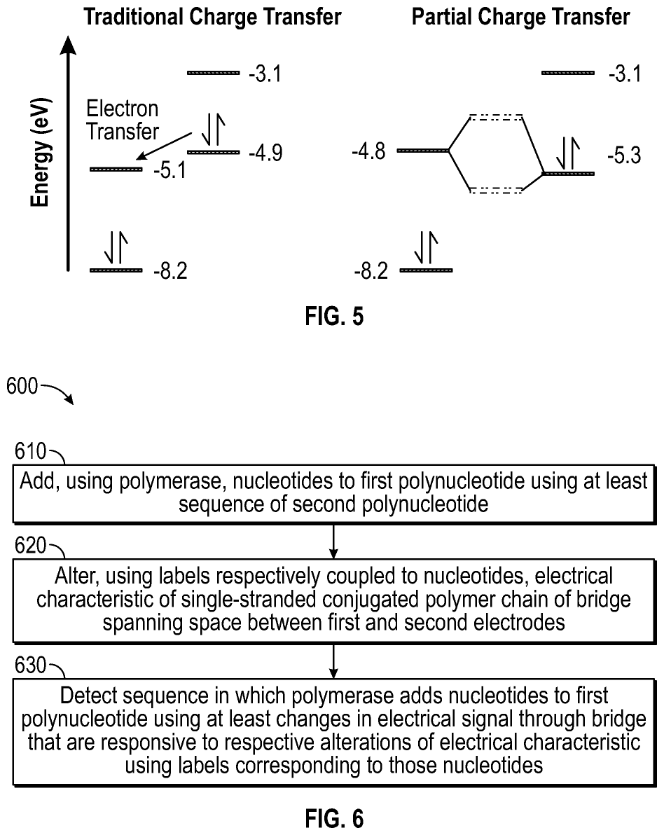

It will be appreciated that in examples such as described with reference to FIGS. 2A-2E, FIGS. 3A-3B, and FIG. 4, the particular configurations of the polymer chain (e.g., the monomer subunit(s) selected and the configuration thereof), as well as the configuration of the labeled nucleotide (e.g., the label and linker) may be selected so as to suitably permit detection of a change in electrical conductivity through the polymer chain caused by association with the label of a labeled nucleotide. For example, the labels may have electron deficient it-cores including fused ring structures that enhance overlap of molecular orbitals, such that formation of a charge-transfer complex with the polymer chain is facilitated by π-π interactions. Electronic properties of a charge-transfer complex may be affected by choice of donors and acceptors. Electronic coupling of the charge-transfer states depends on the difference between the highest occupied molecular orbital (HOMO) energy level of the donor and the lowest unoccupied molecular orbital (LUMO) energy level of the acceptor. For example, FIG. 5 schematically illustrates example charge transfer schemes for altering an electrical characteristic of a single-stranded conjugated polymer bridge using an acceptor labeled nucleotide, in which a favorable energy alignment the LUMO (electron affinity) of the acceptor is lower than the HOMO (ionization potential of the donor (Eoffset<0); while in orbital hybridization the frontier orbitals of the donor and acceptor overlap and split into new orbitals (Eoffset>0). Polythiophene poly(fluorene-alt-thiophene) are nonlimiting examples of conjugated polymer chains that may be included in the present bridges. Rylyene diimides and perylenediimides (PDI) are nonlimiting examples of acceptors that may be included in the present labeled nucleotides, e.g., for use with polythiophene or poly(fluorene-alt-thiophene) polymer chains.

The present polymers may be prepared using any suitable combination of techniques, including synthetic techniques such as described with reference to examples 2 and 4. The length of a conjugated polymer may relate, at least in part, to the molecular weight of the polymer and to the conformation of that polymer in solution (e.g., in an aqueous medium). The molecular weight may be controlled using polymerization parameters such as catalyst loading, monomer concentration, solvent, reaction time, and temperature. The conformation of the polymer may relate to the polarity of the solvent and the nature of the interaction between the polymer and the solvent. Electrostatic repulsion between charges in polyelectrolytes may reduce or inhibit hydrophobic interactions between monomer units and provided a relatively extended polymer conformation which may facilitate bridge formation. As such, in some examples, charged polymers such as DNA may be used as electrostatic templates to modify the conformation of the present conjugated polymer chain in solution, e.g., to cause the present conjugated polymer to obtain an extended conformation in which the end groups of the polymer may contact, and react with, respective electrodes. For example, the present conjugated polymers may be aligned using negatively charged DNA templates. Illustratively, a mixture of DNA and polymer may be stretched on a hydrophobic surface using “molecular combing” to obtain an ordered DNA-polymer complex. Additionally, or alternatively, the present conjugated polymer chains may be electrostatically trapped between the electrodes by applying a voltage between the electrodes to polarize the polymer chains around the electrodes. The polarized polymer chains may be attracted to the area of maximum field between the electrodes.

Compositions such as described with reference to FIGS. 1A-1B, FIGS. 2A-2E, FIGS. 3A-3B, FIG. 4, and FIG. 5 may be used in any suitable method for sequencing. FIG. 6 illustrates an example flow of operations in a method for sequencing using a single-stranded polymer bridge and labeled nucleotides that alter an electrical characteristic of the bridge. In the example illustrated in FIG. 6, the single-stranded polymer of the bridge is conjugated; however, it should be appreciated that the polymer need not necessarily be conjugated. Method 600 includes adding, using a polymerase, nucleotides to a first polynucleotide using at least a sequence of a second polynucleotide (operation 610). For example, polymerase 105 described with reference to FIGS. 1A-1B may add each of nucleotides 121, 122, 123, and 124 to first polynucleotide 160 using at least the sequence of second polynucleotide 150.

Method 600 illustrated in FIG. 6 may include altering, using labels respectively coupled to the nucleotides, an electrical characteristic of a conjugated polymer chain of a bridge spanning a space between first and second electrodes (operation 620). For example, any of labels 131, 132, 133, 134 described with reference to FIGS. 1A-1B respectively may be coupled to nucleotides 121, 122, 123, and 124. As polymerase 105 respectively adds those nucleotides to first polynucleotide 140, the labels coupled to those nucleotides respectively may alter an electrical characteristic of conjugated polymer chain 111 within bridge 110 which spans the space between first electrode 102 and second electrode 103.

Referring again to FIG. 6, method 600 may include detecting a sequence in which the polymerase adds the nucleotides to the first polynucleotide using at least changes in electrical signal, e.g., current or voltage, through the bridge that are responsive to respective alterations of the electrical characteristic using the labels corresponding to those nucleotides (operation 630). For example, detection circuitry 160 described with reference to FIGS. 1A-1B may detect changes in electrical signal through bridge 110 responsive to respective alterations using labels 131, 132, 133, and 134.

### Additional Examples

Additional examples are disclosed in further detail in the following examples, which are not in any way intended to limit the scope of the claims. It will be appreciated that the illustrative reaction schemes set forth in Examples 2 and 4 may be adapted for use in preparing any suitable conjugated polymer chain provided herein. Similarly, it will be appreciated that the illustrative reaction schemes set forth in Example 8 may be adapted for use in preparing any suitable labeled nucleotide provided herein.

### Example 1. Illustrative Single-Stranded Conjugated Polymer

In one nonlimiting example, a single-stranded conjugated polymer, which may act as a donor, had the structure of formula 1:

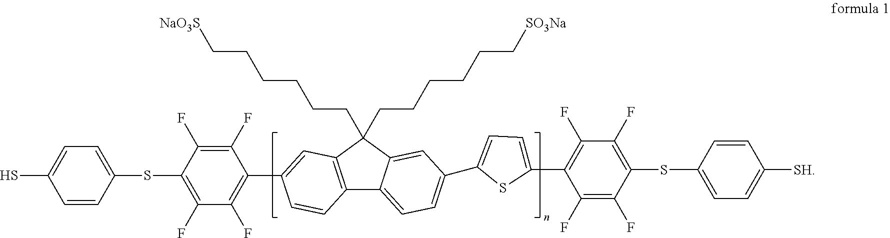

The polymer of formula 1 included a copolymer of monomer units M1 and M2, and a solubilizing group R was coupled to each of the monomer units M1. The starting material for the monomer units M1 of the polymer of formula 1 had the structure:

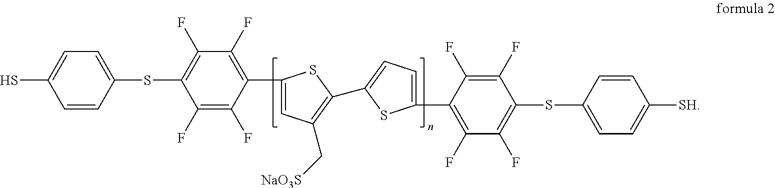

The starting material for the monomer units M2 of the polymer of formula 1 had the structure:

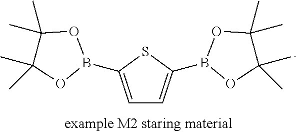

Using the method of Example 2 below, the bromine groups of the starting material for the M1 monomer units were reacted with the boronic acid pinacol ester groups of the starting material for the M2 monomer units, to form an M1-M2 alternating copolymer.

The solubilizing groups R of the polymer of formula 1 had the structure:

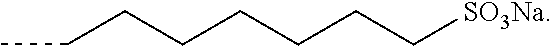

The first and second end groups of the polymer of formula 1 had the structure:

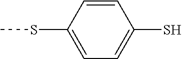

where the dotted line indicates covalent coupling between the thiol and a metal electrode.

### Example 2. Illustrative Synthesis of Single-Stranded Conjugated Polymer of Formula 1

The single-stranded conjugated polymer of Example 1, having the structure of formula 1, was synthesized as follows:

Step 1. Preparation of Starting Material for Monomer Unit M1

The starting material for M1 was synthesized from commercially available precursor A, 2,7-Dibromo-9,9-bis(6-bromohexyl)fluorene (Tokyo Chemical Industries Co. Ltd.). Briefly, A (500 mg, 0.77 mmol), Sodium sulfite (7.7 mmol) and Tetrabutylammonium bromide (0.23 mmol) were added to 20 mL water and refluxed for 1 day. After removing the solvent by evaporation, the residue was washed with methanol several times. The filtrate was dried to give the starting material for monomer unit M1 in 70% yield.

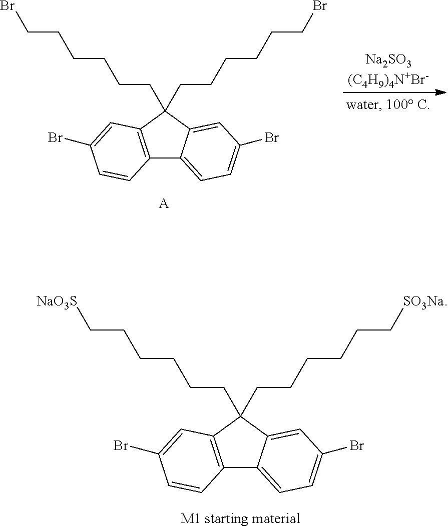

Step 2. Preparation of Starting Material for Monomer Unit M2

The starting material for M2 was obtained commercially from Sigma-Aldrich.

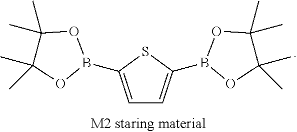

Step 3. Preparation of Polymer Backbone

The polymer backbone was synthesized using a traditional transition-metal catalyzed polycondensation reaction wherein carbon-carbon bonds were formed via coupling of aryl halides and aryl boronate esters. The starting material for monomer M1 was functionalized with terminal bromine groups while the starting material for monomer M2 was functionalized with terminal boronic acid pinacol esters. In the presence of a palladium catalyst and base, the dibrominated monomer of M1 reacts selectively with its diboronate ester coupling partner of M2, to form M1-M2 units with orthogonal terminal groups. The polymer chain propagated by reaction of two or more M1-M2 units with each other or reaction of individual monomers on either end of the M1-M2 unit. This led to the formation of polymers with a distribution of chain lengths.

In order to facilitate the conjugation of end groups to the polymer backbone, as illustrated in Example 1, pentafluorobenzene was added to the terminals of the polymer as a capping group. This was achieved by adding a bromine functionalized reagent G1, along with the starting materials for monomers M1 and M2 at the beginning of the reaction. The stoichiometric ratio of the starting material for M1, the starting material for M2, and G1 was maintained such that total number of orthogonal reactive groups (aryl bromides and aryl boronate esters) are the same. A slight excess of the starting material for monomer M2 compared to the starting material for M1 favors the formation of polymers with pentafluorobenzene-M2 end groups. After sufficient extent of polymerization, a boronic acid pinacol ester functionalized reagent G2 is added to the same pot to cap any polymer chains with the M1 terminal groups. This promotes the formation of a high fraction of polymer chains capped with pentafluorobenzene at both ends. The synthesis procedure of the polymer backbone is described below:

Briefly, the starting material for M1 (0.25 mmol), the starting material for M2 (85.73, 0.255 mmol), G1 (2.5 mg, 0.01 mmol), tetrakis(triphenylphosphine) palladium(0) (14.4 mg, 0.012 mmol), and sodium carbonate (265 mg, 2.5 mmol) were dissolved in a mixture of dry THF (0.5 mL), methanol (1.5 mL), and DI water (0.5 mL) and degassed by applying three freeze-pump-thaw cycles. After stirring the mixture for 36 hours at 85° C. under argon atmosphere, G2 (7.5 mg, 0.0255 mmol) was added and the reaction was continued for 2 hours followed by adding G1 (18.89 mg, 0.0765 mmol) and stirring for another 2 hours. The reaction was cooled down and precipitated in cold THF. The precipitate was filtered through a Soxhlet thimble and washed successively with chloroform and acetone, and finally extracted with methanol. After removing methanol in vacuo the polymer was dried under vacuum to afford an orange powder.

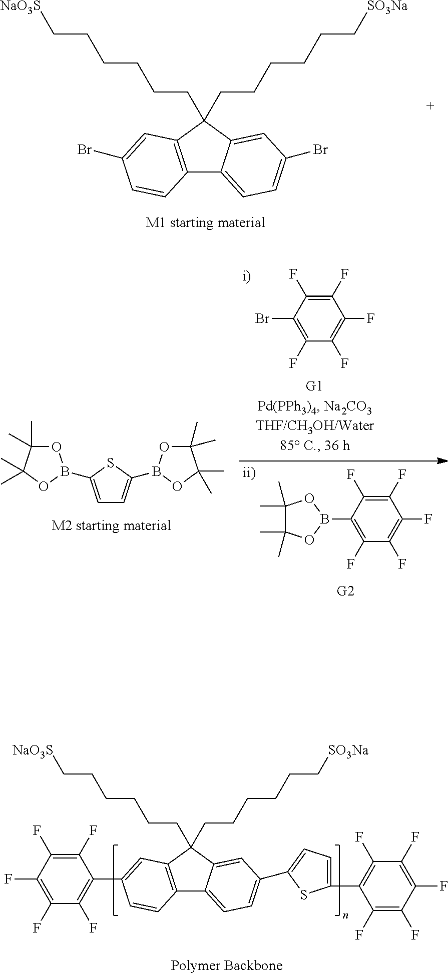

Step 4. Preparation of End Groups

The pentafluorobenzene capping groups of the polymer backbone then were reacted with a compound of formula:

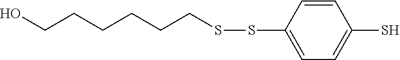 [SMILES: OCCCCCCSSc1ccc(S)cc1]

to complete preparation of the polymer with protected end groups. 1,4-Benzenedithiol was reacted with pentafluorobenzene to introduce the benzenethiol end-group as illustrated in Example 1. However, in order to avoid cross-linking of polymer chains, one thiol group was selectively shielded by forming disulfide bonds with a protecting group.

Briefly, polymer backbone (50 mg), 6-((4-mercaptophenyl) dithio)hexan-1-ol (50 umol) and potassium carbonate (75 umol) were added to methanol (1 mL) and stirred at 70° C. for 12 h. The solution was filtered, and the filtrate was concentrated in vacuo. The solids were washed with chloroform and dried under vacuum to give the polymer with protected end groups.

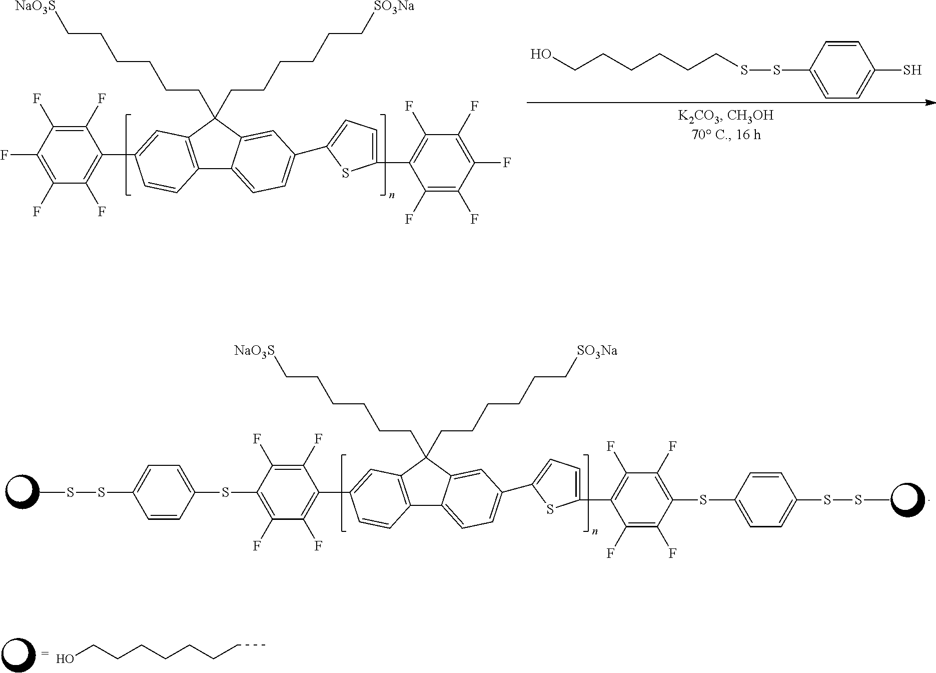

Step 5. Reaction of End Groups with Respective Electrodes

To facilitate reaction of polymer end groups with electrodes, the protected end groups were reduced to obtain free thiol moieties. Briefly, polymer with end groups was incubated in a solution containing 100 molar equivalents of Tris(2-carboxyethyl)phosphine hydrochloride for 4 hours at room temperature to give the polymer of formula 1.

### Example 3. Illustrative Single-Stranded Conjugated Polymer

In one nonlimiting example, a single-stranded conjugated polymer, which may act as a donor, had the structure of formula 2:

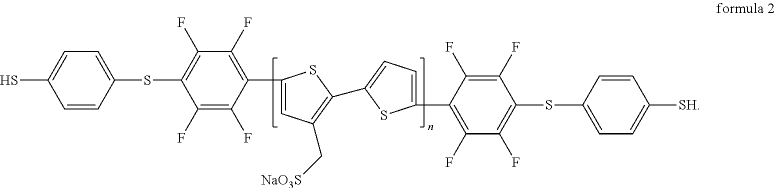

The polymer of formula 2 included a copolymer of monomer units M1 and M2, and a solubilizing group R was coupled to each of the monomer units M1. The starting material for the monomer units M1 of the polymer of formula 2 had the structure:

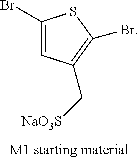

The starting material for the monomer units M2 of the polymer of formula 2 had the structure:

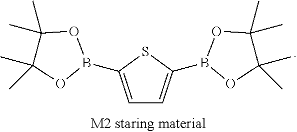

The solubilizing groups R of the polymer of formula 2 had the structure:

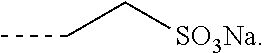

The first and second end groups of the polymer of formula 2 had the structure:

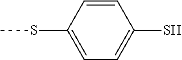

where the dotted line indicates covalent coupling between the thiol and a metal electrode.

### Example 4. Illustrative Synthesis of Single-Stranded Conjugated Polymer of Formula 2

The single-stranded conjugated polymer of Example 3, having the structure of formula 2, was synthesized as follows:

Step 1. Preparation of Starting Material for Monomer Unit M1

The starting material for M1 was prepared in a two-step process from commercially available precursors A, 2-Bromo-3-(bromomethyl) thiophene. Briefly, A (500 mg, 1.95 mmol) and N-Bromosuccinimide (NBS, 2.9 mmol) were added to 10 mL DMF at 0° C. and stirred for 16 h. Solvent was removed by evaporation and crude product was purified by column chromatography to give intermediate B in 80% yield. In the next step, B (500 mg, 1.49 mmol), Sodium sulfite (15 mmol) and Tetrabutylammonium bromide (0.3 mmol) were added to 20 mL of 1:1 water/acetone mixture and refluxed for 1 day. After removing the solvent by evaporation, the residue was washed with methanol several times. The filtrate was evaporated to give M1 starting material in 60% yield.

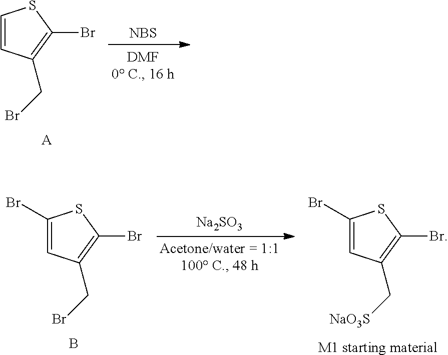

Step 2. Preparation of Starting Material for Monomer Unit M2

The starting material for M2 was obtained commercially from Sigma-Aldrich.

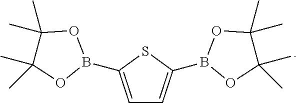

Step 3. Preparation of Polymer Backbone

The polymer backbone was synthesized using a traditional transition-metal catalyzed polycondensation reaction wherein carbon-carbon bonds were formed via coupling of bromine functionalized monomer, the starting material for M1 and boronic acid pinacol ester functionalized monomer, the starting material for M2. Briefly, the starting material for M1 (0.25 mmol), the starting material for M2 (85.73, 0.255 mmol), G1 (2.5 mg, 0.01 mmol), tetrakis(triphenylphosphine) palladium(0) (14.4 mg, 0.012 mmol), and sodium carbonate (265 mg, 2.5 mmol) were dissolved in a mixture of dry THF (0.5 mL), methanol (1.5 mL), and DI water (0.5 mL) and degassed by applying three freeze-pump-thaw cycles. After stirring the mixture for 36 hours at 85° C. under argon atmosphere, G2 (7.5 mg, 0.0255 mmol) was added and the reaction was continued for 2 hours followed by adding G1 (18.89 mg, 0.0765 mmol) and stirring for another 2 hours. The reaction was cooled down and precipitated in cold THF. The precipitate was filtered through a Soxhlet thimble and washed successively with chloroform and acetone, and finally extracted with methanol. After removing methanol in vacuo, the polymer was dried under vacuum to afford a brown powder.

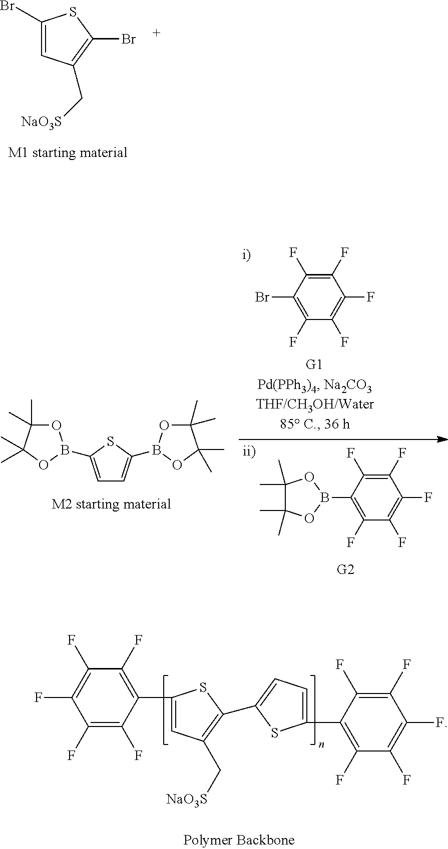

Step 4. Preparation of End Groups

The pentafluorobenzene capping groups then were reacted with a compound of formula:

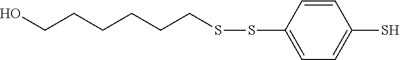 [SMILES: OCCCCCCSSc1ccc(S)cc1]

to complete preparation of the polymer of formula 2. Briefly, polymer backbone (50 mg), 6-((4-mercaptophenyl) dithio)hexan-1-ol (50 umol) and potassium carbonate (75 umol) were added to methanol (1 mL) and stirred at 70° C. for 12 h. The solution was filtered, and the filtrate was concentrated in vacuo. The solids were washed with chloroform and dried under vacuum to give the polymer with protected end groups.

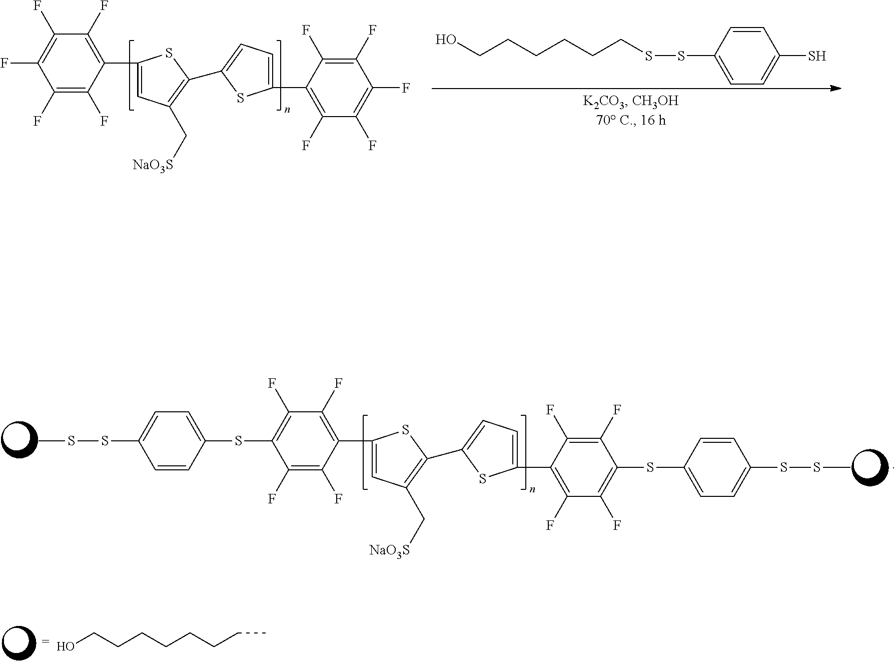

Step 5. Reaction of End Groups with Respective Electrodes

To facilitate reaction of the polymer end groups with electrodes, the protected end groups were reduced to obtain free thiol moieties. Briefly, polymer with —OH end groups was incubated in a solution containing 100 molar equivalents of Tris(2-carboxyethyl)phosphine hydrochloride for 4 hours at room temperature to give the polymer of formula 2.

### Example 5. Illustrative Acceptor-Labeled Nucleotide

In one nonlimiting example, an acceptor-labeled nucleotide, which may form respective charge-transfer complexes with the polymers of formula 1 and formula 2, has the structure of formula 3:

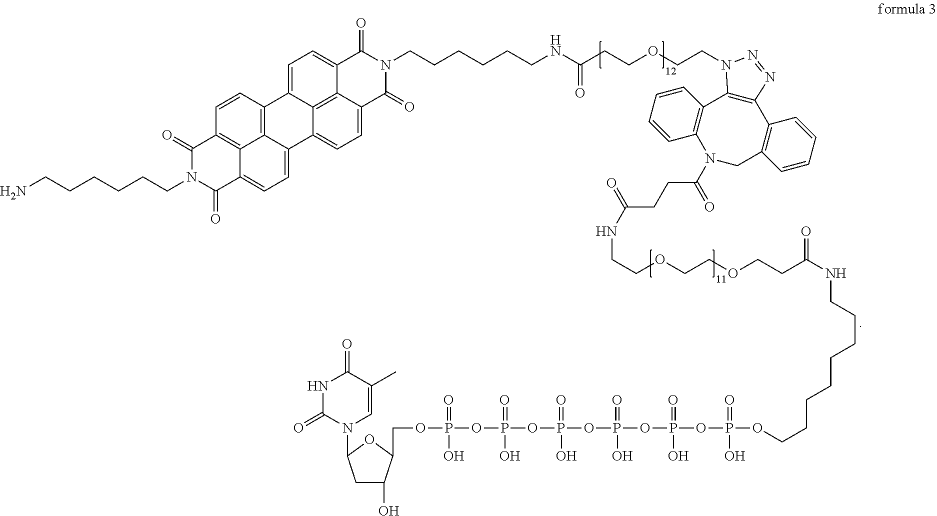

The acceptor-labeled nucleotide of formula 3 may include a nucleotide coupled to an acceptor via a linker. The nucleotide dT6P of the acceptor-labeled nucleotide of formula 3 has the structure:

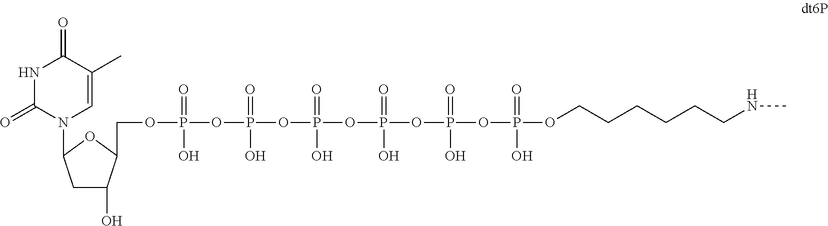

where the dotted line indicates coupling of the nucleotide to the linker.

The linker of the acceptor-labeled nucleotide of formula 3 has the structure:

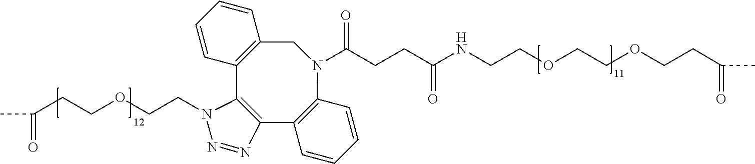

where the dotted lines indicate respective couplings of the linker to the acceptor and the nucleotide.

The acceptor of the acceptor-labeled nucleotide of formula 3 have the structure:

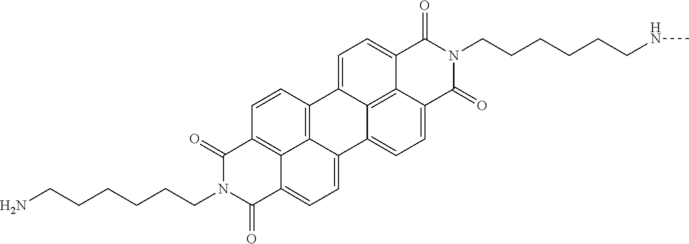

where the dotted line indicates coupling of the acceptor to the linker.

### Example 6. Illustrative Acceptor-Labeled Nucleotide

In one nonlimiting example, an acceptor-labeled nucleotide, which may form respective charge-transfer complexes with the polymers of formula 1 and formula 2, has the structure of formula 4:

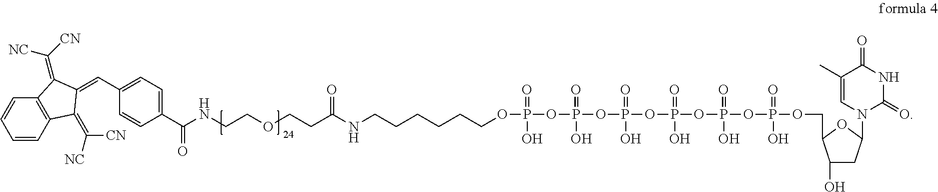

The acceptor-labeled nucleotide of formula 4 may include a nucleotide coupled to an acceptor via a linker. The nucleotide dT6P of the acceptor-labeled nucleotide of formula 4 has the same structure as for the acceptor-labeled nucleotide of formula 3. The linker of the acceptor-labeled nucleotide of formula 4 has the below structure, where the dotted lines indicate respective attachments to the acceptor and to the nucleotide:

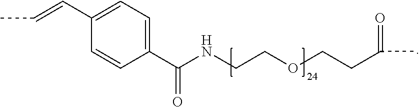

The acceptor of the acceptor-labeled nucleotide of formula 4 has the structure:

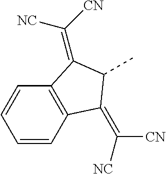

where the dotted line indicates coupling of the acceptor to the linker.’

### Example 7. Illustrative Acceptor-Labeled Nucleotide

In one nonlimiting example, an acceptor-labeled nucleotide, which may form respective charge-transfer complexes with the polymers of formula 1 and formula 2, has the structure of formula 5:

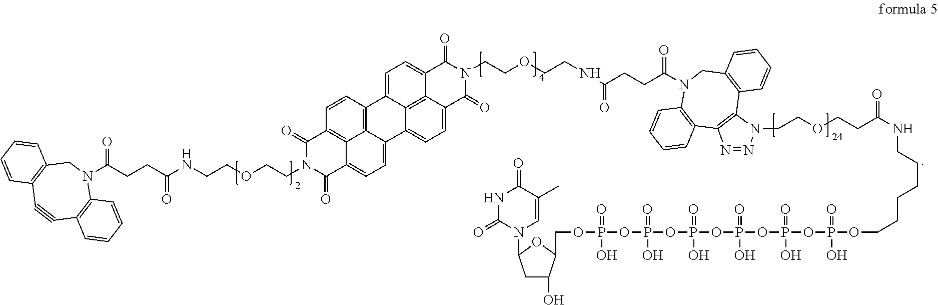

The acceptor-labeled nucleotide of formula 5 may include a nucleotide coupled to an acceptor via a linker. The nucleotide of the acceptor-labeled nucleotide of formula 5 has the structure:

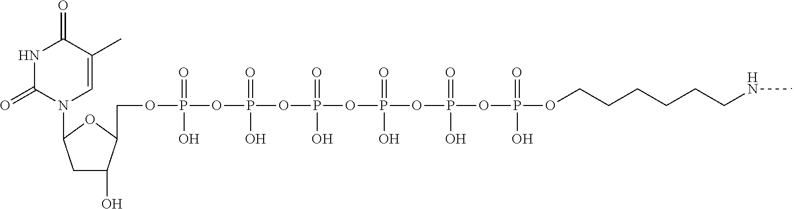

The starting material of the linker of the acceptor-labeled nucleotide of formula 5 may have the structure:

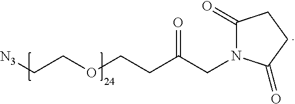

The starting material of the acceptor of the acceptor-labeled nucleotide of formula 5 may have the structure:

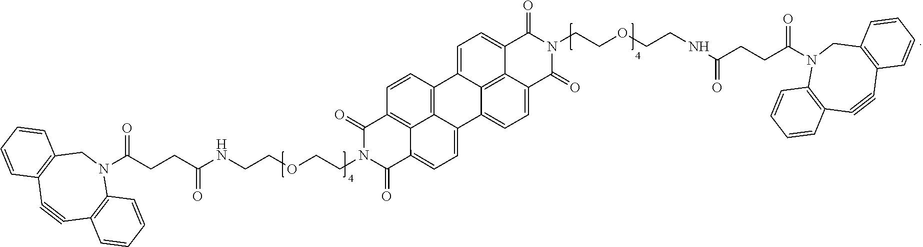

### Example 8. Illustrative Synthesis of Acceptor-Labeled Nucleotide of Formula 5

The acceptor-labeled nucleotide of example 7, having the structure of formula 5, may be synthesized as follows:

Step 1. Preparation of Acceptor

The acceptor is synthesized from commercially available precursors A, Perylene-3,4,9,10-tetracarboxylic dianhydride (Sigma Aldrich) and B, DBCO-PEG4-amine (BroadPharm). Briefly, A (100 mg, 0.25 mmol), B (0.75 mmol) and imidazole (3.75 mmol) are added to a flask and heated at 130° C. for 2 days under argon atmosphere. The crude products are dissolved in dichloromethane and washed with 1 M HCl and water. The organic phase is concentrated, and the residue was purified by column chromatography to give the acceptor as a red solid in expected 40% yield.

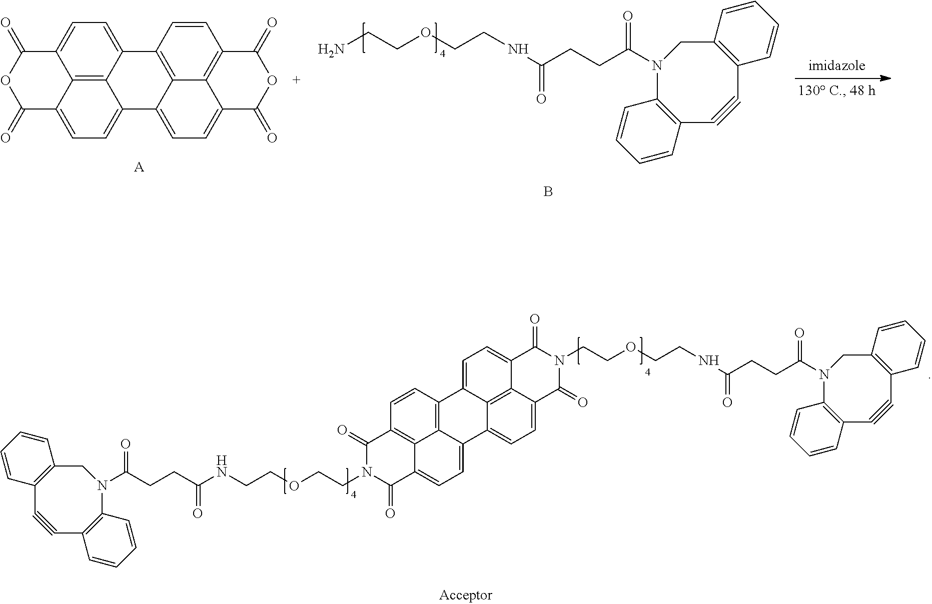

Step 2. Preparation of Nucleotide

The nucleotide used in this example is commercially obtained from MyChem LLC.

Step 3. Preparation of Acceptor-Labelled Nucleotide

Acceptor-labelled nucleotide is prepared by a two-step process wherein the first step involves addition of the linker group, Azido-PEG24-NHS (BroadPharm) to the nucleotide. The second step involves conjugation of nucleotide-linker unit to the acceptor.

Briefly, nucleotide (1.6 mg, 2 umol) and Azido-PEG24-NHS (5 umol) are mixed in 1× phosphate-buffered saline solution, pH 7.4 at room temperature for 16 hours. The crude product is purified by reverse phase chromatography to yield nucleotide-linker unit in expected 50% yield.

Nucleotide-linker unit (2 mg, 1 umol) and acceptor (1 umol) are stirred in 1× phosphate-buffered saline solution, pH 7.4 at room temperature for 16 hours. The crude product is purified by reverse phase chromatography to yield the acceptor-labeled nucleotide of formula 5 in expected 40% yield.

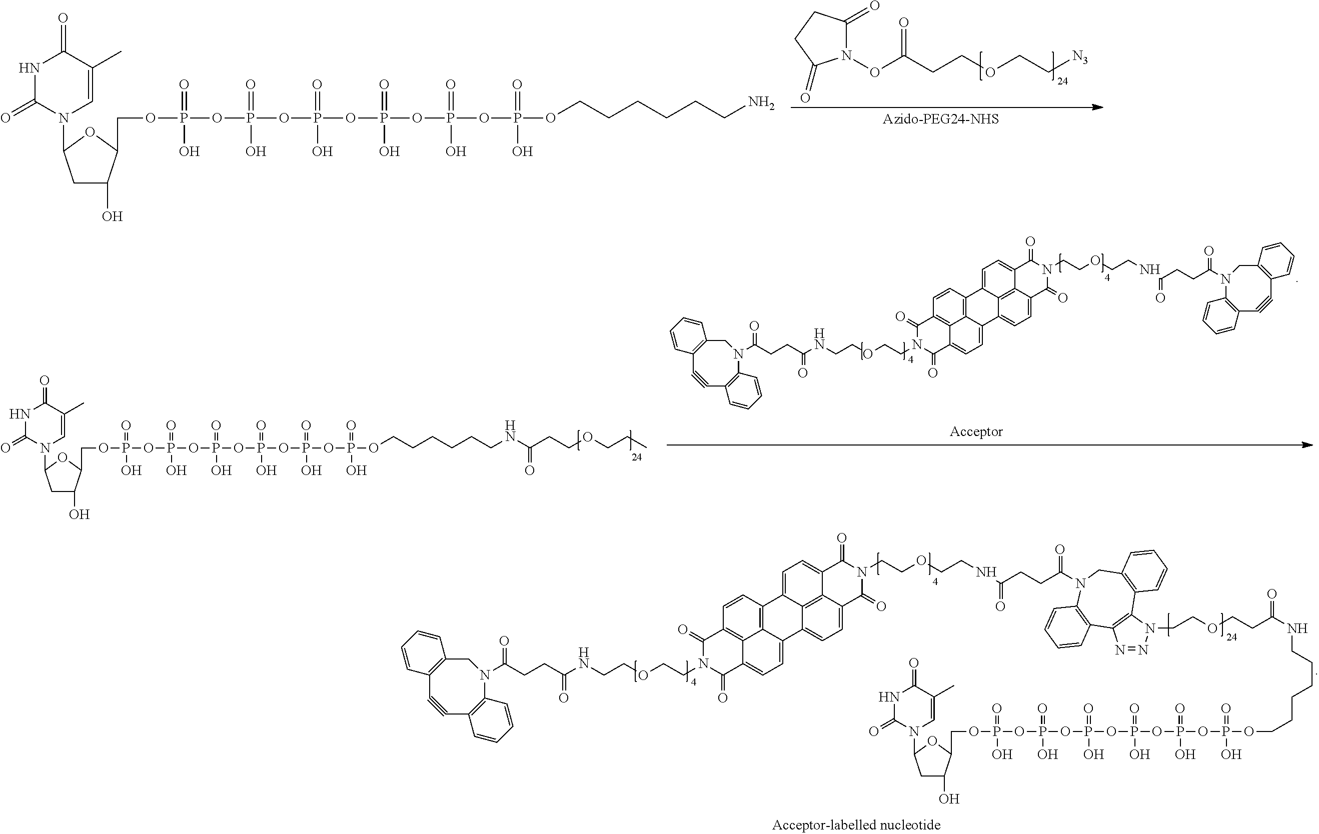

Accordingly, it may be understood from Examples 1, 2, 3, and 4 that various conjugated polymer bridges may be prepared, e.g., using reaction schemes such as described in Examples 2 and 4. Additionally, it may be understood from Examples 5, 6, 7, and 8 that various acceptor-labeled nucleotides may be prepared, e.g., using reaction schemes such as described in Example 8.

### Additional Examples

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A composition, comprising:
first and second electrodes separated from one another by a space;
a bridge spanning the space between the first and second electrodes, the bridge comprising a single-stranded conjugated polymer chain;
first and second polynucleotides;
a plurality of nucleotides, each nucleotide coupled to a corresponding label;
a polymerase to add nucleotides of the plurality of nucleotides to the first polynucleotide using at least a sequence of the second polynucleotide,
the labels corresponding to those nucleotides respectively altering an electrical characteristic of the single-stranded conjugated polymer chain; and
detection circuitry to detect a sequence in which the polymerase adds the nucleotides to the first polynucleotide using at least changes in an electrical signal through the bridge, the changes being responsive to alteration of the electrical characteristic using the labels corresponding to those nucleotides.

2. The composition of claim 1, wherein the single-stranded conjugated polymer chain comprises a first delocalized set of orbitals, and each of the labels comprises a respective delocalized set of orbitals that shares electrons with the first delocalized set of orbitals when the label is associated with the single-stranded polymer chain.

3. The composition of claim 1, wherein each of the labels and the single-stranded conjugated polymer chain forms a donor:acceptor complex.

4. The composition of claim 1, wherein the labels comprise respective acceptors, the single-stranded conjugated polymer chain comprises a donor, and electrons transfer from the single-stranded polymer chain to each of the labels via π-π interactions.

5. The composition of claim 1, to wherein the labels include different molecular orbitals and detectably change electrical conductivity of the single-stranded conjugated polymer chain differently.

6. The composition of claim 1, wherein the single-stranded polymer chain comprises a homopolymer.

7. The composition of claim 6, wherein the homopolymer comprises repeating monomer units M1, each of the monomer units M1 being coupled to a solubilizing group R.

8. The composition of claim 1, wherein the single-stranded polymer chain comprises a copolymer comprising first monomer units M1 and second monomer units M2, each of the first monomer units M1 being coupled to a solubilizing group R.

9. The composition of claim 8, wherein the copolymer comprises repeating M1-M2 units.

10. The composition of claim 1, wherein the single-stranded polymer chain comprises a copolymer comprising first monomer units M1 and second monomer units M3, wherein (i) each of the first monomer units M1, or (ii) each of the second monomer units M3, or (iii) each of the first monomer units M1 and each of the second monomer units M3, is coupled to a solubilizing group R.

11. The composition of claim 10, wherein the copolymer comprises repeating M1-M3 units.

12. The composition of claim 1, wherein the single-stranded polymer chain comprises a copolymer comprising first monomer units M3 and second monomer units M2, each of the first monomer units M3 being coupled to a solubilizing group R.

13. The composition of claim 12, wherein the copolymer comprises repeating M3-M2 units.

14. The composition of claim 1, wherein the single-stranded polymer chain comprises a copolymer comprising first monomer units M1, second monomer units M2, and third monomer units M3, wherein (i) each of the first monomer units M1, or (ii) each of the second monomer units M3, or (iii) each of the first monomer units M1 and each of the second monomer units M3, is coupled to a solubilizing group R.

15. The composition of claim 14, wherein the copolymer comprises repeating M1-M2 units.

16. The composition of claim 14, wherein the copolymer comprises repeating M3-M2 units.

17. The composition of claim 7, wherein M1 is an electron-donating or electron-neutral monomer unit that includes the solubilizing group R, and wherein the electron-donating or electron-neutral monomer unit that includes the solubilizing group R is selected from the group consisting of: 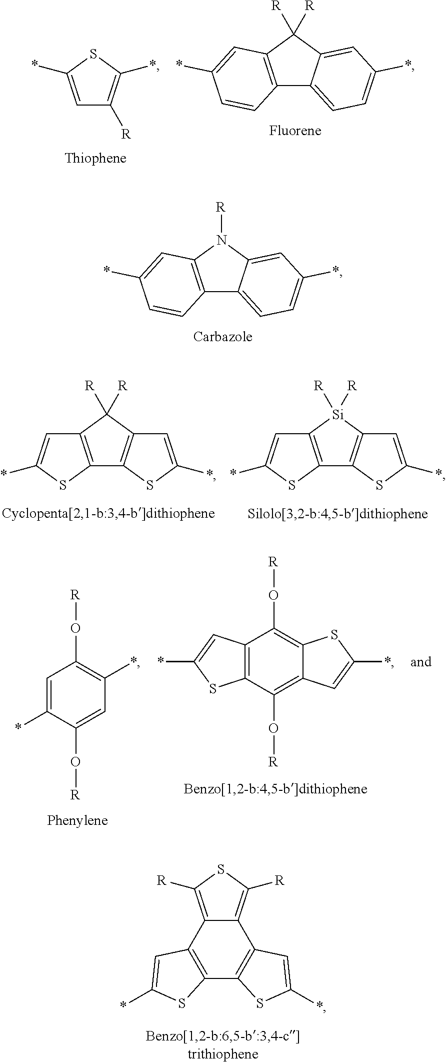
where * indicates coupling to an end group or to another monomer unit.

18. The composition of claim 7, wherein R is a cationic solubilizing group selected from the group consisting of: (N,N,N-Trimethyl ammonium) alkyl, (N,N,N-Tributyl ammonium)alkyl, 1-methyl-3-(alkyl)-Imidazolium, and 1-(alkyl)Pyridinium.

19. The composition of claim 7, wherein R is an anionic solubilizing group selected from the group consisting of: alkyl sulfonate, alkyl carboxylate, and alkyl phosphonate.

20. The composition of claim 7, wherein M2 is an electron-donating monomer unit that excludes the solubilizing group R, and wherein the electron-donating monomer unit that excludes the solubilizing group R is selected from the group consisting of: 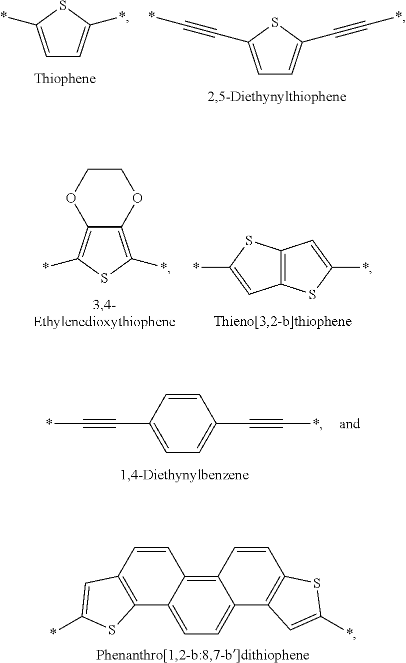
where * indicates coupling to an end group or to another monomer unit.

21. The composition of claim 7, wherein M3 is an electron-withdrawing monomer unit that excludes the solubilizing group R, and wherein the electron-withdrawing monomer unit that excludes the solubilizing group R is selected from the group consisting of: 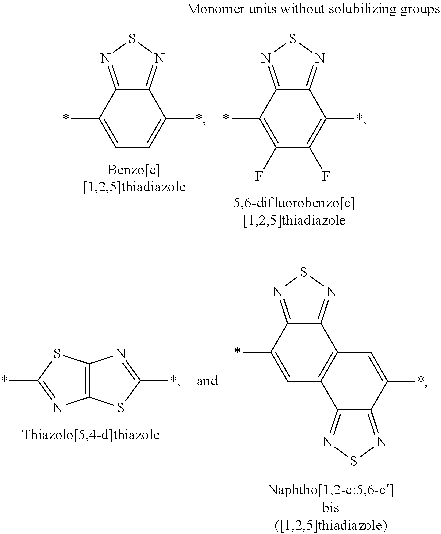
where * indicates coupling to an end group or to another monomer unit.

22. The composition of claim 7, wherein M3 is an electron-withdrawing monomer unit that includes the solubilizing group R, and wherein the electron-withdrawing monomer unit that includes the solubilizing group R is selected from the group consisting of: 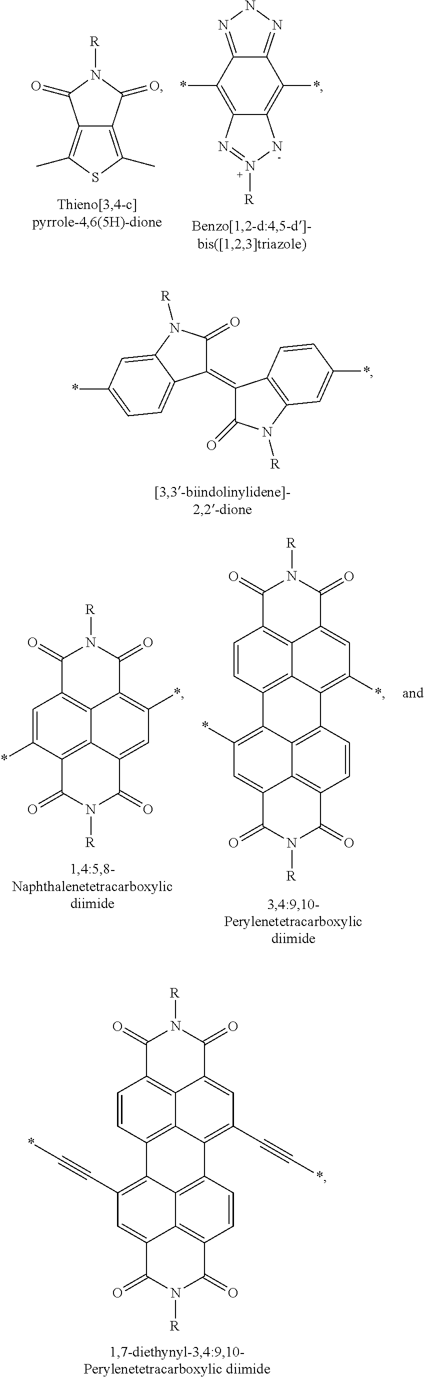
where * indicates coupling to an end group or to another monomer unit.

23. The composition of claim 1, further comprising a first end group coupling the single-stranded conjugated polymer chain to the first electrode, and a second end group coupling the single-stranded conjugated polymer to the second electrode.

24. The composition of claim 23, wherein each of the first and second end groups is selected from the group consisting of: Benzenethiol, Phenylacetylene, Benzenediazonium, Benzylamine, Methyl benzyl sulfide, and Methyl hexyl sulfide.

25. The composition of claim 1, wherein the labels are acceptors selected from the group consisting of: 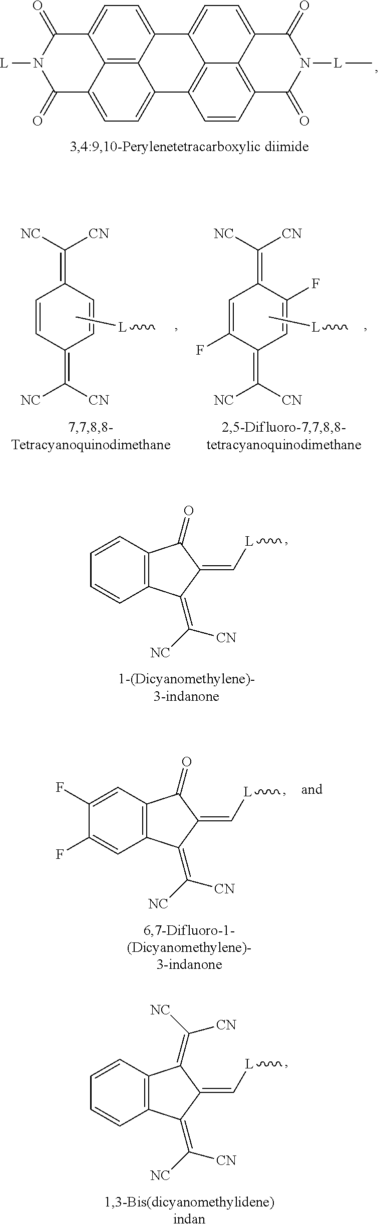
in which L indicates a linker and the wavy line represents coupling of linker L to the nucleotide.

26. A method, the method comprising:
adding, using a polymerase, nucleotides to a first polynucleotide using at least a sequence of a second polynucleotide;
altering, using labels respectively coupled to the nucleotides, an electrical characteristic of a single-stranded conjugated polymer chain of a bridge spanning a space between first and second electrodes; and
detecting a sequence in which the polymerase adds the nucleotides to the first polynucleotide using at least changes in electrical signal through the bridge that are responsive to respective alterations of the electrical characteristic using the labels corresponding to those nucleotides.

27-50. (canceled)
